SEQUENCE LISTING (33 sequences)

SEQ ID NO: 1 - DNA, 27 bp, Artificial Sequence
  DNA fragment from Hepattitis B virus
ttggcagcacaccctagcagccatgga

SEQ ID NO: 2 - DNA, 15 bp, Artificial Sequence
  DNA fragment from Hepattitis B virus
gatgactgtctctta

SEQ ID NO: 3 - DNA, 15 bp, Artificial Sequence
  DNA fragment from Hepattitis B virus
aggagattgacgaga

SEQ ID NO: 4 - DNA, 15 bp, Artificial Sequence
  DNA fragment from Hepattitis B virus
gttctccatgttcgg

SEQ ID NO: 5 - DNA, 12 bp, Artificial Sequence
  DNA fragment from Hepattitis B virus
tctccatgttcg

SEQ ID NO: 6 - DNA, 15 bp, Artificial Sequence
  DNA fragment from Hepattitis B virus
gaatcctgatgtaat

SEQ ID NO: 7 - DNA, 15 bp, Artificial Sequence
  DNA fragment from Hepattitis B virus
aacatgagggaaaca

SEQ ID NO: 8 - DNA, 23 bp, Artificial Sequence
  DNA fragment from Hepattitis C virus
tgctcatggtgcacggtctacga

SEQ ID NO: 9 - DNA, 20 bp, Artificial Sequence
  DNA fragment from Hepattitis C virus
ctttcgcgacccaacactac

SEQ ID NO: 10 - DNA, 21 bp, Artificial Sequence
  DNA fragment from Hepattitis C virus
catgatgcacggtctacgaga

SEQ ID NO: 11 - DNA, 20 bp, Artificial Sequence
  DNA fragment from Hepattitis C virus
gcctttcgcgacccaacact

SEQ ID NO: 12 - DNA, 17 bp, Artificial Sequence
  DNA fragment from Hepattitis C virus
gcctttcgcgacccaac

SEQ ID NO: 13 - DNA, 17 bp, Artificial Sequence
  DNA fragment from Hepattitis C virus
gcctttcgcgacccaac

SEQ ID NO: 14 - DNA, 20 bp, Artificial Sequence
  DNA fragment from Hepattitis C virus
gtgctcatggtgcacggtct

SEQ ID NO: 15 - DNA, 16 bp, Artificial Sequence
  DNA fragment from Hepattitis C virus
gtgctcatggtgcacg

SEQ ID NO: 16 - DNA, 20 bp, Artificial Sequence
  DNA fragment from Hepattitis C virus
ctgctcatggtgcacggtct

SEQ ID NO: 17 - DNA, 15 bp, Artificial Sequence
  DNA fragment from Hepattitis D virus
gcggcagtcctcagt

SEQ ID NO: 18 - DNA, 15 bp, Artificial Sequence
  DNA fragment from Hepattitis D virus
ctcggctagaggcgg

SEQ ID NO: 19 - DNA, 15 bp, Artificial Sequence
  DNA fragment from Hepattitis D virus
ctcggaccggctcat

SEQ ID NO: 20 - DNA, 15 bp, Artificial Sequence
  DNA fragment from Hepattitis D virus
tcttccgaggtccgg

SEQ ID NO: 21 - DNA, 17 bp, Artificial Sequence
  DNA fragment from Hepattitis D virus
atatcctatggaaatcc

SEQ ID NO: 22 - DNA, 15 bp, Artificial Sequence
  DNA fragment from Hepattitis D virus
tgagtggaaacccgc

SEQ ID NO: 23 - DNA, 17 bp, Artificial Sequence
  DNA fragment from Hepattitis D virus
atttgcaagtcaggatt

SEQ ID NO: 24 - RNA, 15 nt, Artificial Sequence
  RNA fragment from hepatoma-derived cells
agucagucagucagu

SEQ ID NO: 25 - RNA, 15 nt, Artificial Sequence
  RNA fragment from hepatoma-derived cells
guucuccauguucag

SEQ ID NO: 26 - RNA, 21 nt, Artificial Sequence
  RNA fragment from hepatoma-derived cells
uuuauaagggucgauguccau

SEQ ID NO: 27 - DNA, 15 bp, Artificial Sequence
  Antiviral oligonucleotide
gttctccatgttcag

SEQ ID NO: 28 - DNA, 21 bp, Artificial Sequence
  Antiviral oligonucleotide
tttataagggtcgatgtccat

SEQ ID NO: 29 - DNA, 16 bp, Artificial Sequence
  Control oligomer
aaagccacccaaggca

SEQ ID NO: 30 - DNA, 22 bp, Artificial Sequence
  DNA fragment non-complementary from Hepatitis B virus
tgagctatgcacattcagattt

SEQ ID NO: 31 - DNA, 14 bp, Artificial Sequence
  DNA fragment non-complementary from Hepatitis B virus
tccaattagatcag

SEQ ID NO: 32 - RNA, 15 nt, Artificial Sequence
  RNA fragment from hepatoma-derived cells modified in 2′ position
agucagucagucagu

SEQ ID NO: 33 - DNA, 12 bp, Artificial Sequence
  Radiolabelled DNA fragment from Hepatitis B virus
ntcctcctgcgg

(19) United States
(12) Patent Application Publication
(10) Pub. No.: US 2006/0183886 A1
(43) Pub. Date: Aug. 17, 2006

(54) Ligands to enhance cellular uptake of biomolecules

(75) Inventors: Paul O.P. Tso, Ellicott City, MD (US); Robert Duff, York, PA (US); Yuanzhong Zhou, Columbia, MA (US); Scott Deamond, Baltimore, MD (US); Clinton Roby, Baltimore, MD (US)

Correspondence Address: FISH & RICHARDSON PC, P.O. BOX 1022, MINNEAPOLIS, MN, 55440-1022, US

(73) Assignee: Cell Works Therapeutics, Inc., a Delaware corporation

(21) Appl. No.: 11/256,476

(22) Filed: Oct. 21, 2005

Related U.S. Application Data
(62) Division of application No. 09/888,164, filed on Jun. 22, 2001, now abandoned.
Application No. 09/888,164 is a continuation of application No. 09/282,455, filed on Mar. 31, 1999, now abandoned.
Application No. 09/282,455 is a continuation-in-part of application No. 08/755,062, filed on Nov. 22, 1996, now Pat. No. 5,994,517.
(60) Provisional application No. 60/007,480, filed on Nov. 22, 1995.

(51) Int. Cl. C07K 14/005; C07K 16/10

(52) U.S. Cl. 530/350; 530/388.3

(57) ABSTRACT

The present invention relates to the design and synthesis of homogeneous A-L-P constructs, which contain a hepatic ligand to direct an oligomer or “payload” to a hepatocyte intracellularly via a receptor-mediated, ligand-directed pathway.

### FIELD OF THE INVENTION

This invention relates to the delivery of biodegradation-resistant, homogeneous oligonucleoside conjugates to cells in a tissue specific manner via ligand directed, receptor mediated, endocytosis pathway.

### BACKGROUND OF THE INVENTION

The liver is a vital organ and is responsible for many biological functions. Some of its most important functions include detoxifying and excreting substances that otherwise would be poisonous, processing nutrients and drugs from the digestive tract for easier absorption, producing bile to aid in is the digestion of food, and converting food into chemicals for life-sustaining growth and maintenance. At least 100 different types of liver diseases are known. The most important diseases of the liver are viral hepatitis, cirrhosis, and cancer.

Currently, there are five known types of viral hepatitis: hepatitis A virus (HAV), hepatitis B virus (HBV), hepatitis C virus (HCV), hepatitis D virus (HDV), and hepatitis E virus (HEV). However, based on epidemiological studies, other hepatropic viruses appear to exist because hepatitis A-E viruses fail to account for all known cases.

HAV and HEV are spread through contaminated food and water, but do not cause chronic liver disease. In contrast, HBV, HDV, and HCV are bloodborne viruses that may lead to chronic infection and chronic hepatitis. Two of the most important liver viruses are HBV and HCV. HBV is estimated to infect 320,000 individuals annually (Centers for Disease Control, unpublished data), and that there are about 1 to 1.25 million HBV persons with a chronic infection (Seroprevalence data from the Third National Health and Nutrition Examination Survey (NHANES III, 1996). Worldwide estimates suggest that there are 200 million people infected with HBV. HDV is a defective virus and requires co-infection with HBV or a preexisting (superinfection) infection of HBV (Smedile et al., (1981), Gastroenterology, 81:992-997) both of which elicits more severe symptoms than a HBV infection alone. A chronic HDV infection in an individual infected with HBV is associated with high liver failure. An HCV infection is estimated to afflict 3.5 million carriers with about 150,000 new infections annually. An HCV infection is accompanied by mild symptoms and may not be diagnosed until the development of chronic disease. About 80% of HCV infections become chronic and lead to liver disease. Drug therapy, such as interferon therapy, is aimed at reducing inflammation, symptoms, and infectivity, but rarely is the virus eliminated in infected individuals.

According to the National Cancer Society, in 1998, new cases of hepatocellular carcinoma (HCC) were responsible for 13,900 deaths in the United States. Annual deaths due to HCC range from 500,000 to 1 million people worldwide. HCC is widespread in Southeast Asia, particularly Hong Kong. Individuals with hepatitis B, C, D or liver cirrhosis are at a greater risk of developing HCC. Chronic viral hepatitis may causel cirrhosis of the liver, hepatocellular failure and HCC. Currently, there is no known efficacious treatment against HCC.

Malaria is a disease caused by a number of protozoan parasites from the genus Plasmodium and is spread by female mosquitos of the genus Anopheles. The four species of Plasmodium that cause malaria are P. vivax, P. ovale, P. malariae, and P. falciparum. The disease most commonly occurs in the tropics and subtropics, such as Central America, South America, Southeast Asia, the Carribbeans, the South Pacific Islands, and sub-Saharan Africa Symptoms appear anywhere from a week to a month after the mosquito bite, and include high fever, shaking chills, sweats, headache, muscle aches, fatigue, anemia, and sometimes vomiting and coughing. The most severe form of malaria is characterized by fever, confusion, spleen enlargement, nausea, and anemia, and can be fatal. If the disease is left untreated, the infection will progress to fluid in the lungs, liver failure, kidney failure, brain swelling, coma, and death. The lifecycle of the parasite that causes malaria begins when a female mosquito bites an infected human ingesting some gametocytes, which undergo meiosis and mature in the mosquito's stomach. As a result, male and female gametes fuse to form a zygote that migrates within the mosquito and develops to produce sporozoites in the salivary glands of the mosquito. These sporozoites infect the blood of the next human host, and ultimately get the host's liver. Eventually, some parasites leave the liver and begin to reproduce in the blood cells of the host, which leads to the familiar symptoms of malaria.

One approach to treating viral and protozoan infections, and cancer is harnessing the power of antisense therapies. The selective inhibition of gene expression through specific oligonucleotide binding to vital mRNA target sequences is the major goal in applying antisense technology to the regulation of DNA and proteins. The selective inhibition of gene expression through specific oligonucleotide binding to vital mRNA target sequences is the major goal in applying antisense technology to the regulation of the genetic elements, such as RNA, DNA, and proteins. The antisense (anticode or antigene) strategy for drug design is based on the sequence-specific inhibition of protein synthesis by the delivery of synthetic oligodeoxynucleotides (oligo-dN) and their analogs that are able to bind and mask the target mRNA or genomic DNA (Mirabelli, et al., (1993), In Antisense Research and Applications, (Crooke and LeBleu, Eds.), CRC Press, Boca Raton, pp. 7-35). Implicit in this strategy is the ability of oligo-dNs to cross cellular membranes, thereby gaining access to the cellular compartments containing their intended targets, and to do so in sufficient amounts for binding to those targets to take place.

Delivery of exogenous DNA into the intracellular medium is greatly enhanced by coupling its uptake to receptor-mediated endocytosis. Pioneering work by Wu and Wu ((1987), J. Biol. Chem., 262:4429-4432) showed that foreign genes (Wu, (1987), supra; Wu, (1988), J. Biol. Chem., 263:14621-14624; Wu, (1988), Biochemistry, 27:887-892) or oligo-dNs ((1992), J. Biol. Chem., 267:12436-12439), electrostatically complexed to poly-L-lysine linked to asialoorosomucoid, are efficiently and specifically taken into human hepatocellular carcinoma (Hep G2) cells through direct interaction with the asialoglycoprotein receptor. Since this initial study, other examples of receptor-mediated delivery of DNA have appeared in the literature, including a tetra-antennary galactose neoglycopeptide poly-L-lysine conjugate (Plank, et al., (1992), Bioconjugate Chem., 3:533-539); a trigalactosylated bis-acridine conjugate (Haensler, et al., (1993), Bioconjugate Chem., 4:85-93); folate conjugates (Kamen, et al., (1988), J. Biol. Chem., 263:13601-13609); an antibody conjugate (Trubetskoy, et al., (1992), Bioconjugate Chem., 3:323-327); transferrin conjugate (Wagner, et al., (1990), Proc. Natl. Acad. Sci. USA, 87:3410-3414); and a 6-phosphomannosylated protein linked to an antisense oligo-dNs via a disulfide bond (Bonfils, et al., (1992), Nucleic Acids Res., 20:4621-4629). Recently, the tri-antennary N-acetylgalactosamine neoglycopeptide, YEE(ahGalNAc)₃ (Lee and Lee (1987), Glycoconjugate J., 4:317-328), was conjugated to human serum-albumin and then linked to poly-L-lysine, was shown to deliver DNA into Hep G2 cells (Merwin, et al., (1994), Bioconjugate Chem., 5:612-620).

A number of products have been described for the delivery of oligo-dNs, which are heterogeneous mixtures of conjugates. Bonfils et al., for example, describe formation of conjugates between 6-phosphomannosylated protein and oligonucleosides which, because the modification of the protein and the formation of the disulfide link are not regiochemically controlled, or site-specific, yields a heterogeneous mixture of structurally different molecules (Bonfils, supra).

Several studies have described intracellular delivery of oligodeoxynucleotides or DNA, which contain biodegradable phosphodiester internucleotide linkages. Because of the inherent susceptibility of phosphodiesters to hydrolysis, payload constructs containing biogradeable internucleotide linkages may have relatively short half lives within the cell and efficacy is consequently reduced (Wickstrom (1986), J. Biochem. Biophys. Meth. 13:97-102). For example, an all phosphodiester 16-mer was extensively 10 degraded after a few minutes in the cell (Shaw, et al., (1991), Nucleic Acids Research, 19:747-750). This disadvantage with oligo-dNs and DNA is well recognized in the antisense community.

Merwin et al. describe the synthesis of conjugates using the neoglycopeptide YEE(ahGalNAc)₃. Their delivery system is heterogeneous and is contains poly-L-lysine, which serves to electrostatically bind DNA to the conjugate. The disadvantages of this delivery strategy are: its structural heterogeneity; potential toxicity due to its polycationic charge; and difficulties in formulation due to the need to empirically determine the ratio of cationic carrier to oligo-dn or DNA for optimum delivery.

The use of antisense oligonucleotides and their analogs as therapeutic agents has been complicated by their lack of specific delivery and limited cellular uptake leading to low intracellular concentrations (Loke et al., (1989), Proc. Natl. Acad. Sci., 86:3474-3478; Levis et al., (1995), Antisense Res. & Dev., 5:251-259. Enhanced cellular uptake of these molecules has been achieved by coupling their delivery to receptor mediated endocytosis utilizing various structurally heterogeneous complexes (Plank et al., (1992), Bioconj. Chem., 3:533-539; Kamen et al., (1988), J. Biol. Chem., 263:13602-13609; Wagner et al., (1990), Proc. Natl. Acad. Sci., 87:3410-3414). A number of these complexes have been shown to deliver small molecules specifically to the liver cells in vitro at an enhanced rate via the hepatic asialoglycoprotein receptor, (ASGP-R) (Wu and Wu., (1997), J. Biol. Chem., 262:4429-4432; Findeis et. al., (1994), Mtds. Enzymol., 247:341-351. These complexes include those formulated with a tri-antennary, N-acetylgalactosamine neoglycopeptide, YEE(ahGalNAc)₃, which displays a high affinity for the mammalian ASGP-R (Lee and Lee, (1987), Glycoconj. J., 4:317-328; Merwin et. al., (1994), Bioconj. Chem., 5:612-620. We have previously demonstrated that covalent conjugation of methylphosphonate oligomers (OMNP) to this neoglycopeptide via a structurally defined and heterobifunctional linker resulted in enhanced and specific cellular uptake by hepatoma cells in vitro (Hangeland et al, (1995), Bioconj. Chem., 6:695-701, as well as specific delivery to the liver of mice in vivo (Hangeland et al., (1997), Antisense & Nuc. Acid Drug Dev., 7:141-149.

Among the many oligo-dN analogs for application as antisense, non-ionic oligonucleoside methylphosphonates (oligo-MPs) have been extensively studied (Ts'o, et al., (1992), Ann. NY Acad. Sci, 600:159-177). Oligo-MPs are totally resistant to nuclease degradation (Miller, et al., (1981), Biochemistry, 20:1874-1880) and are effective antisense agents with demonstrative in vitro activity against herpes simplex virus type 1 (Smith, et al., (1986), Proc. Natl. Acad. Sci. USA, 83:2787-2791), vesicular stomatitis virus (Agris, et al., (1986), Biochemistry, 25:6269-6275) and human immunodeficiency virus (Sarin, et al., (1988), Proc. Natl. Acad. Sci. USA, 85:7448-7451), and are able to inhibit the expression of ras p21 (Brown, et al., (1989), Oncogene Res., 4:243-252). For oligo-MPs to exhibit antisense activity, however, they must be present in the extracellular medium in concentrations up to 100 μM (Brown, supra; Sarin, supra; Ts'o, supra; Agris, supra). Achieving and maintaining these concentrations for therapeutic purposes presents a number of difficulties, including expense, potential side effects owing to non-specific binding of the oligo-MP and immunogenicity. These difficulties can be circumvented by enhancing transport of the oligo-MP across the membrane of the targeted cell types, thereby achieving a locally high concentration of the oligo-MP, and by specific delivery to a target cell type only, thereby avoiding toxic side effects to other tissues. Both strategies serve to greatly reduce the concentration of the oligo-MP needed to produce an antisense effect and to avoid the toxic side effect with tissue specificity.

The present invention overcomes such deficiencies by delivering A-L-P constructs that are homogeneous and are non-biodegradeable, which serves to deliver potent therapies to a target cell intracellularly for enhanced effective and/or non-toxic effects.

### SUMMARY OF THE INVENTION

It is an object of the invention to design and synthesize a homogeneous A-L-P construct containing a hepatic ligand to direct an oligomer or “payload” to a hepatocyte intracellularly via a receptor-mediated, ligand-directed pathway.

It is another object of the invention to deliver a stable payload or oligomer directed to a liver pathogen via a A-L-P construct to a hepatocyte. The liver pathogen may be a virus, a parasite, or cancer.

It is another object of the invention to provide a structurally defined and chemically uniform delivery assembly, which consists of ligand-linker-pro-drug construct, that is directed to hepatocytes via a ligand directed, receptor-mediated endocytotic pathway.

It is a further object of the invention to provide a homogeneous construct to a molecular target within a cell Comprising the delivery of an A-L-P construct containing a biologically non-degradable “P”, or a hydrolytic enzyme resistant pro-drug, wherein said pro-drug contains oligo dN and/or oligo dN analogs, which can efficiently cross hepatocyte's membranes and gain access to the cytoplasm.

Another object of the invention is to deliver an assortment of DNA and RNA types of payload, e.g., payloads containing methylphosphonates, phosphodiesters, and phosphorothioates linkages of DNA and methylphosphonate-2′-O-methylribose, phosphodiester-2′-O-methylribose, and phosphorothioate-2′-O-methylribose moieties of RNA.

Another object of the invention concerns the delivery of a payload intracellularly to a target cell, which may contain combinations of internucleotide linkages of varying degrees of biodegradeability upon entry to a cell target, such linkages include methylphosphonates/phosphodiesters (mp/po) linkages, phospho-diesters/phosphorothioates (po/ps) linkages and methylphosphonates/phosphorothioates (mp/ps) linkages for DNA; and methyl-phosphonate/phosphodiesters-2′-O-methylribose (mp/po-OMe), phosphodiesters/phosphorothioates-2′-O-methylribose (po/ps-OMe), methylphosphonates/phosphorothioates-2′-O-methylribose (mp/ps-OMe) for RNA. A preferred object of the invention is to deliver oligodeoxynucleoside phosphothioroate conjugates, which contain enzymatically-resistant phosphorothioate internucleotide linkages, to hepatocytes. Another preferred object of the invention is to deliver oligodeoxynucleoside methylphosphonate conjugates, which contain non-biodegradable methylphosphonate internucleotide linkages, to hepatocytes. The delivery of biologically stable oligomers, such as non-ionic oligodeoxynucleoside and oligonucleoside analogs, intracellularly to hepatocytes containing a hepatic virus and/or cancer is a means of treating the liver pathogen. In particular, it is a further object of the invention to provide the delivery of synthetic conjugates of oligodeoxynucleoside chimeras that contain all 2′-O-methylribose nucleosides and internucleotide linkages that alternate between methyl-phosphonate and phosphodiester or any other biostable oligomers. Such biostable oligomers include, but are not limited to, oligodeoxynucleotide analogs that contain: all 2′-deoxyribose nucleosides and internucleotide linkages that alternate between phosphorothioate and methylphosphonate; all 2′-deoxyribose nucleosides and phosphorothioate internucleotide linkages; all 2′-O-methylribose and phosphorothioate internucleotide linkages.

Another object of the invention concerns methods for synthesizing A-L-P conjugates. One particular method for synthesizing conjugates comprises a three-component Conjugation Method 1 for the synthesis of A-L-P conjugates, wherein
a) a 2′-O-Me-nucleotide phosphodiester linkage is incorporated to the 5′-end of the oligonucleotide or oligonucleotide analogs;
b) the 5′-end of the oligonucleotide or oligonucleotide analog is enzymatically phosphorylated using PNK and ATP;
c) the 5′-phosphate group of the oligonucleotide or oligonucleotide analog is modified to introduce a disulfide linkage to form 5′-disulfide-modified oligonucleotide or oligonucleotide analog;
d) the 5′-disulfide group of the 5′-disulfide-modified oligonucleotide or oligonucleotide analog is reduced to a thiol group to form a thiol-modified oligonucleotide; and
e) one reactive group of the heterobifunctional linker is covalently conjugated to a ligand and a second group of the heterobifunctional linker is covalently conjugated to said thiol-modified oligonucleotide or oligonucleotide analogs to form the A-L-P conjugate.

Another method concerns the synthesis of conjugates comprises a Conjugation Method 2 for the synthesis of an A-L-P conjugate, wherein
a) a ligand is modified w % ith a bifunctional linker to form an A-L construct;
b) said A-L construct is purified to greater than 95% homogeneity and to remove unreacted linker;
c) the oligonucleotide or oligonucleotide analog is modified to form a thiol-modified oligomer;
d) said thiol-modified oligomer is purified under degassed conditions;
e) a conjugation reaction using a purified A-L construct and a purified thiol-oligomer in a two-component conjugation reaction is executed under degassed conditions to remove unreacted reagent and other low molecular weight thiol-containing impurities; wherein said conjugation can be performed by using either excess-amounts of said ligand scaffold or said thiol-modified oligomer to form purified A-L-P conjugates; and
f) the A-L-P conjugate is purified, for example, by chromatography or electrophoresis.

Another method concerns radiolabeling an oligonucleotide-containing conjugate, comprising radiolabeling an A-L-P conjugate, wherein
a) a tri-nucleotide tracer unit, 5′-T-3′-ps-3′-T-ps-T-5′ is added to the 3′-end of an oligonucleotide or an oligonucleotide analog during solid-phase synthesis;
b) said tracer unit is subjected to enzymatic phosphorylation using PNK and ATP to form a modified tracer unit; and
c) said modified tracer unit is chemically modified with an amine of the radioactive phosphate group of the A-L-P conjugate to prevent cellular enzymatic degradation.

Another method concerns the synthesis of oligonucleotide-containing conjugates wherein
a) a bifunctional linker terminating in a disulfide moiety is incorporated onto an oligonucleotide or an oligonucleotide analog during solid-phase synthesis to form a disulfide modified oligomer;
b) said disulfide-modified oligomer is purified;
c) the disulfide moiety of said disulfide-modified oligomer is reduced to a thiol group to form a thiol-modified oligomer;
d) said thiol-modified oligomer is purified using size exclusive chromatography under degassed conditions;
e) a conjugation reaction using a purified A-L and a purified thiol-oligomer is executed under degassed conditions to form an A-L-P conjugate; and
f) the synthesized A-L-P conjugate is purified, for example, by chromatography.

Another method concerns the synthesis of a radiolabeled conjugate comprising the radiolabel of A-L-P conjugates containing an oligonucleotide or an oligonucleotide analog; wherein
a) a disulfide linker is incorportated into the 5′-end and a trinucleotide tracer unit, 5′-T-3′-ps-3′-T-ps-T-5′, at the 3′-end of the oligonucleotide analogs during solid-phase synthesis;
b) the disulfide- and tracer-containing oligomer is purified;
c) the disulfide is reduced to a thiol group to form a thiol-modified oligomer;
d) said thiol-modified oligomer is purified, for example, using size exclusion chromatography under degassed conditions;
e) a purified A-L is conjugated to a purified thiol-oligomer under degassed conditions to form an A-L-P conjugate;
f) the tracer unit is enzymatically phosphorylated to incorporate a radiolabeled phosphate into the A-L-P conjugate using PNK and radiolabeled ATP; and
g) the radioactive phosphate group of the ATP conjugate is chemically modified with an amine to protect it from cellular enzymatic degradation.

### BRIEF DESCRIPTION OF THE DRAWINGS

FIG. 1 shows the attachment groups for chemically uniform conjugates. The value of n is between 0 and 10, inclusive (Compounds 1-4).

FIG. 2 shows the structures of neoglycopeptide YEE(ahGalNAc)₃ (5) (FIG. 2a); oligo-MP U^{m}pT₇ (6), and 5′-ethylenediamine capped U^{m}pT₇ (6b) (FIG. 2b); Structure of the Tracer, 3′ conjugate (FIG. 2c); Reaction scheme for the automated synthesis with 5′-thiol modifier (FIG. 2d); and Reaction scheme for the synthesis of 1c (FIG. 2e).

FIG. 3 depicts a reaction scheme for the synthesis of [YEE(ahGalNAc)₃]-SMCC-AET-pU^{m}pT₇. (10).

FIG. 4 shows PAGE analysis (15% polyacrylamide, 4 V/cm, 2 h) of intermediates in the synthesis of conjugate 10. Lane 1, [5′-³²P]-labeled 6 (band A). Lane 2, [5′-³²P]cystamine adduct (band B) and corresponding thymidine-EDAC adducts (bands C). Lane 3, [5′-³²P]-thiol 5 (band D) and corresponding thymidine-EDAC adducts (bands E). Lane 4, [5′-³²P]-conjugate 10 (band F) and corresponding thymidine-EDAC adducts (bands G).

FIG. 5 illustrates the structures of the [³⁵S]3′-End Labeled hepatitis B virus (HBV) neoglycoconjugates.

FIG. 6 shows a time course for the uptake by Hep G2 cells of 1 μM conjugate 10, alone (open circles) and in the presence of 100 equivalents of free 5 (closed circles), and oligo-MP 11, alone (open triangles) and in the presence of 10 equivalents of free 5 (closed triangles). Cells with incubated at 37° C. for 0, 1, and 2 hours and samples collect as described in the experimental section. Each data point represents the average of three trials±one standard deviation.

FIG. 7 shows a 24 hour time course for the uptake of conjugate 10 by Hep G2 cells. Cells were incubated at 37° C. and the cells collected as described in the experimental section. Each data point represents the average of three experiments±one standard deviation.

FIG. 8 shows the tissue specific uptake of conjugate 10 by Hep G2, HL-60 and HT 1080 cells Cells were collected and the amount of [³²P] determined at 3 and 24 h for each cell line. Experiments were done in triplicate and the data expressed as the average±one standard deviation.

FIG. 9 shows the uptake of neoglycoconjugates containing nuclease resistant backbones 10 by Hep G2 cells.

FIG. 10 shows the uptake of neoglycoconjugates containing nuclease resistant backbones 10 by Hep G2 2.2.15 cells.

FIG. 11 shows the tissue distribution of conjugate 10, which was produced by removing the terminal GalNAc residues of conjugate 10 with N-acetyl-glucosamidase—Percent initial dose per gram tissue versus time post-injection for conjugate 10.

FIG. 12 shows the tissue distribution of conjugate 12, which was produced by removing the terminal GalNAc residues of conjugate 10 with N-acetyl-glucosamidase. Percent initial dose per gram tissue versus time post-injection for conjugate 12.

FIG. 13 (a) shows the tissue distribution of a S³⁵-labeled antisense phosphorothiate-containing neoglycoconjugate in mice; (b) shows the tissue distribution in mice of a S³⁵-labeled antisense phosphorothiate-containing neoglycoconjugate that has the terminal galNAc removed. Values are reported as the average of three trials±one SD. It was assumed that blood is approximately 7% and muscle is 40% of the body weight.

FIG. 14 shows the autoradiographic analysis of the metabolites of 10 in Hep G2 cells. Lane 1, 1; Lane 2, 1 treated with N-acetyl-glucosaminidase; Lane 3, 1 treated with chymotrypsin; lanes 4-8; Hep G2 cell extracts following incubation with 1 for 2, 4, 8, 16, and 24 hours respectively.

FIG. 15 shows the autoradiographic analysis of the metabolites of 10 in mouse liver. Lane 1, 1; Lane 2, 1 treated with N-acetyl-glucosaminidase; Lane 3, 1 treated with chymotrypsin; Lane 4, treated with 0.1 N HCl; Lanes 5-9, liver homogenate extracts at 2 hours, 1 hour and 15 minutes post injection. Note that lanes 5 and 6 are replicates as well as lanes 7 and 8.

FIG. 16 shows a structure of 10. The conjugate was synthesized with radioactive phosphate located on the 5′-OH of the oligoMP moiety. The arrowhead marks the position of the ³²P label. Structure of 10 written in abbreviated form. Structures 11 and 12-15 are proposed structures of metabolites identified by PAGE analysis. Structures 12-15 obtained by treating 10 with N-acetylglucosamine, chymotrypsin or 0.1 HCl, respectively.

FIG. 17 shows the autoradiographic analysis of the metabolites of 10 in mouse urine. Lane 1, 1; Lane 2, 1 treated with N-acetyl-glucosaminidase; Lane 3, 1 treated with chymotrypsin; Lane 4, treated with 0.1 N HCl; Lanes 5-8, urine extractions at 2 hours, 1 hour and 15 minutes post injection. Note that lanes 5 and 6 are replicates.

FIG. 18 shows the effect of anti-HBV neoglycoconjugates on the accumulation of HBsAG in the culture media of HepG2 2.2.15 cells: A. Anti-S; B. Anti-C; C. Anti-E; Solid bars=Untreated controls Stippled bars=Neoglycoconjugates; Crosshatched Bars=Unconjugated oligomers.

FIG. 19 shows the effect of anti-HBV neoglycoconjugates on the accumulation of HBV virion DNA in the culture media of HepG2 2.2.15 cells: A=Anti-S; B=Anti-C; C=Anti-E; Solid bars=Untreated controls; Stippled bars=Neoglycoconjugates; Crosshatched Bars=Unconjugated oligomers.

FIG. 20 shows the effect of random neoglycoconjugate and oligomers on: HBsAG and HBV virion DNA accumulation in the media of Hep G2 2.2.15 cells in culture. A=Effect of NG-4 on HBs AG accumulation; B=Effect of NG-5 and corresponding oligomer on HBsAG accumulation; C=Effect of NG-4 and corresponding oligomer on HBV virion DNA accumulation; D=Effect of NG-5 corresponding oligomer on HBV virion DNA accumulation; Solid bars=untreated controls, Stippled bars=neoglycoconjugate, Cross-hatched bars=unconjugated oligomers.

Abbreviations

For convenience, the following abbreviations are used: AET, 2-aminomercaptoethanol (aminoethanethiol); ATP, adenosine triphosphate; BAP, bacterial alkaline phosphatase; CPG, controlled pore glass support; DIPEA, diispropylethylamine; D-MEM, Dulbecco's modified-Eagle's medium; DMSO, dimethyl sulfoxide; D-PBS, Dulbecco's phosphate buffered saline; DTT, dithiothreitol; EDAC, 1-ethyl-3-[3(dimethylamino)propyl]carbodiimide; EDTA, ethylenediaminetetraacetate; FCS, fetal calf serum; GalNAc, N-acetylgalactosamine; MEM, minimal essential medium with Earle's salts; SMCC, N-hydroxysuccinimidyl 4 (N-methylmaleimido)cyclohexyl-1 carboxylate; Tris, tris(hydroxymethyl)amine; PNK, phenylnucleotidekinase.

### DETAILED DESCRIPTION OF THE INVENTION

The invention is directed to the design and synthesis of a homogenenous molecular construct designated as a ligand-linker-pro-drug construct or an “A-L-P” construct, wherein “A” represents a ligand that specifically binds to a cellular receptor; “P” represents a “payload”; and “L,” is a defined molecular bridge that unites the ligand and the pro-drug through its linkage, wherein “A” and “P” are covalently attached to the linker; and further, the A-L-P construct delivers the “payload”, to the specific cell target, such as a hepatocyte, through a receptor-mediated, ligand-directed, endocytic pathway.

In a preferred embodiment, the A-L-P construct acts as a delivery system, which comprises a homogeneous conjugate of formula A-L-P, wherein “A” represents a ligand that specifically binds to a hepatic receptor, thereby facilitating the entrance of said conjugate into cells having said receptor; “L” rep resents a bifunctional linker that is chemically combined with A in a regiospecific manner to form A-L; A-L is chemically combined with P in a regiospecific manner to form A-L-P; “P” represents a biologically stable oligomer, such as an oligonucleotide or oligonucleotide derivative, wherein P is released from the conjugate following hydrolysis or reduction of specific biochemical linkages and contains internucleotide linkages resistant to enzymatic hydrolysis or biodegradation upon release from the conjugate.

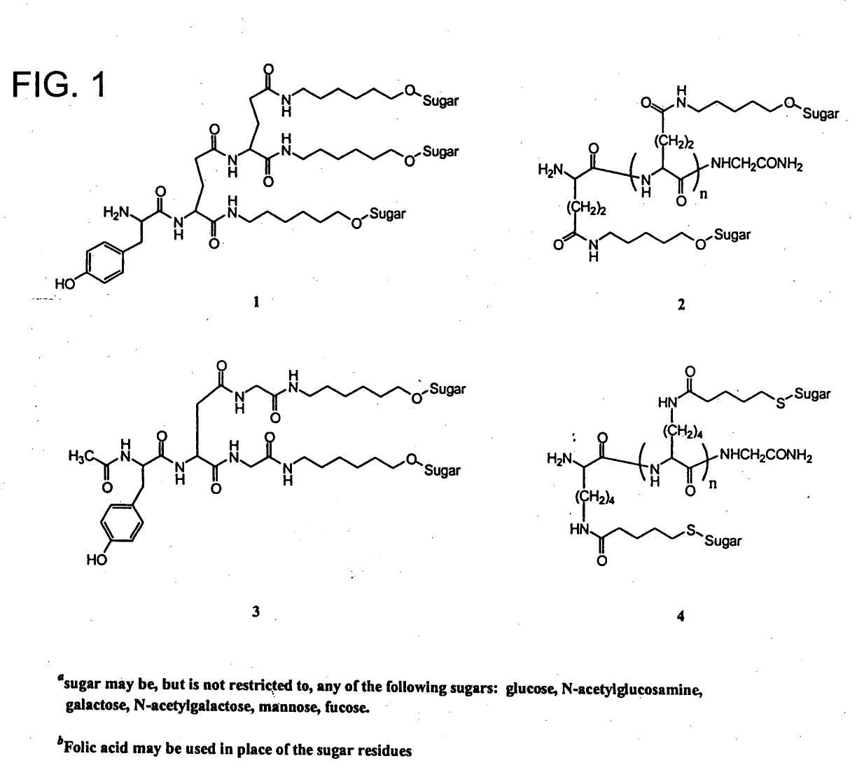

The linkages between the ligand and linker and the linker and pro-drug are covalent, and are formed through a cross-linking reagent, which is capable of forming covalent bonds with the ligand and the pro-drug. A wide variety of cross-linking reagents are available that are capable of reacting with various functional groups present on the ligand and the pro-drug, thus, many chemically distinct linkages can be constructed. For example, the ligand YEE(ahGalNAc)₃ (FIG. 1, 1) contains a free amino group at its amino terminus. It will react regiospecifically with the heterobifunctional cross-linking reagent, SMCC (Table 4; entry 3), to form an amide bond. The pro-drug, if chemically modified to contain a free sulhydryl group (Table 2; for examples see entries 9-14) will chemically combine with SMCC to form a thioether linkage. In this example, the linkage formed between the ligand and pro-drug could be summarized as amide/thioether. It is apparent that hundreds of structures can be formulated by combining the ligands, cross-linking reagents and pro-drugs (FIG. 1; and illustrated in Tables 1-4) in all of the possible combinations. Thus other linkages include, but are not restricted to, amide/amide, thioether/amide, disulfide/amide, amide/thioether, amide/disulfide. The linkages can be further categorized as biologically stable (thioether, amine), somewhat biologically stable (amide), and biologically labile (disulfide). Thus, the delivery system can be modified structurally to function in the various chemical environments encountered in the extra- and intracellular medium. The ligands for this delivery system include, but are not restricted to those shown in FIG. 1. The term “attachment groups”, as used herein, refers to these and other suitable ligands. The ligands consist of a synthetic, chemically defined, structurally homogeneous oligopeptide scaffold that is glycosylated with any of a number of sugar residues including, but not restricted to: glucose; N-acetylglucosamine; galactose; N-acetylgalactosamine; mannose; and fucose.

The term “neoglycopeptide”, as used herein, refers to these and similar structures. In addition, these oligopeptides provide frameworks to construct multivalent ligands with folic acid.

The term “pro-drug”, as used herein, means a compound that, upon hydrolysis or bioreduction of specific chemical linkage(s), is released from the conjugate to become active or more active than when contained as part of the conjugate.

The term “chemically uniform”, as used herein, means that at least 95% of the delivery assembly, and most preferably 99%, is a single species both in composition and in connectivity. Determination of chemical uniformity is by polyacrylamide gel electrophoresis, reverse-phase high pressure liquid chromatography, nuclear magnetic resonance, mass spectrometry and chemical analysis. The phrase “chemically defined and structurally homogeneous” is used interchangeably with “chemically uniform”.

The term “efficiently”, as used herein, is intended to mean that, for example, if the conjugate is present in the extracellular medium, then following a 24 hour incubation period at 37° C., the intracellular concentration will be at least approximately 3 times and preferably approximately 10 times the extracellular concentration.

The term “oligomer” is used within the context of this invention to include oligonucleotides, oligonucleotide analogues, or oligonucleosides, or is also known as the “payload” or upon entry to the cell it may also include the conversion of a pro-drug to a drug. The term “oligonucleotide analog” shall mean moieties that have at least one non-naturally-occurring portion, and which function similarly to or superior to naturally-occurring oligonucleotides. Oligonucleotide analogues may have altered sugar moieties or altered inter-sugar linkages. An oligonucleotide analogue having at least one non-phosphodiester bond, such as an altered inter-sugar linkage, can alternately be considered an “oligonucleoside.” These oligonucleosides refer to a plurality of nucleoside units joined by linking groups other than naturally-occurring phosphodiester linking groups. An oligonucleotide analog encompasses analogs that contain at least one “non-phophodiester internucleotide bond, i.e., a linkage other than a phosphodiester between the 5′ endo of a one nucleotide and the 3′ end of another nucleotide in which the 5′ nucleotide phosphate has been replaced with any number of chemical groups. Preferably, the oligomer of the A-L-P construct is directed to a hepatic pathogen, wherein such pathogen comprises any disease-causing mircoorganism or process, such as a virus, parasite, and cancer. More preferably, the virus may comprise a hepatitis virus, such as hepatitis A virus (HAV), hepatitis B virus (HBV), hepatitis C virus (HCV), hepatitis D virus (HDV), and hepatitis E virus (HEV). In particular, sequences targeted directly to a viral surface antigen, a core antigen, an open reading frame, and an encapsidation sequence are an object of the invention. For example, hepatitis B virus comprises a hepatitis B a surface antigen, S-gene, core antigen, C-gene, preS1 open reading frame, and virus encapsidation signal/sequence. In addition, the parasite may comprise a plasmodium.

Different linkages-backbones, such as methylphosphonate (mp) (all non-ionic), alternating methylphosphonate/phosphodiester (mp/po), (half-charged), and phosphorothioates (ps) (fully charged), have different characteristics including different charges as indicated in the parentheses. Other oligonucleotides with other nuclease-resistant backbones include phosphorothiates (ps) and oligomers comprised of 2′O methylribose moieties with an alternating phosphodiester/methylphosphonate (po/mp) linkages. Preferable synthetic linkages include alkylphosphonates, phosphorothioates, phosphorodithioates, alkylphosphorothioates, phosphoramidates, phosphoroamidites, phosphate esters, carbamates, carbonates, phosphate triesters, acetamidate, and carboxymethyl esters. Any of these linkages may also be substituted with various comical groups, e.g., an aminoalkylphosphate. In one preferred embodiment of the invention, all of the nucleotides of the oliogonucleotide are linked via phosphorothioate and/or phosphor odithioate linkages. The preparation of such linkages use known methodologies (Meth. Mol. Biol., Vol. 20 (Agrawal, ed.), Humana Press, New Jersey; Uhlmann, supra).

An oligonucleotide shall mean a polymer of several nucleotide residues. In particular, the term “oligonucleotide”, as used herein, has the meaning as ordinarily used in the art, e.g., a linear sequence of up to 50 nucleotides (“50 mer”) or more preferably a sequence of 15 to 30 nucleotides, and most preferably, about 20 nucleotides (“20 mer”). The oligonucleotides utilized in the invention are often, but not always, antisense oligonucleotides, which are oligonucleotides having a sequence which is complementary to a particular cellular or foreign DNA or RNA within the target cells. Such molecules also include ribozymes, which shall mean RNA molecules with catalytic activities, including, but not limited to, the ability to cleave at specific phosphodiester linkages in RNA molecules to which they have hybridized, such as mRNAs, RNA-containing substrates, and ribozymes. Generally, if “P” is an antisense oligonucleotide, the preferred molecular weight is about 5,000 to 10,000 Daltons; and the most preferred molecular weight is about 5,000 to 7,500 Daltons. An antisense RNA shall mean an RNA molecule that binds to a complementary mRNA molecule, forming a double-stranded region that inhibits is translation of the mRNA. Generally, in this particular scenario, the molecular weight of the linker of the present invention is less than or equal to the molecular weight of the “P” antisense.

Preferably, oligomers of the present invention comprise linkages that are non-biodegradeable, and more specifically, any nuclease-resistant backbone including ones that are fully or partially resistant. Examples of these oligomers include chimeric oligonucleotides, which comprise internal phosphodiester and terminal methylphosphonodiester linkages (Giles, et al. (1992), Anticancer Drug Des, 7:31-48), such as methylphosphonodiester/phosphodiester chimeric antisense oligodeoxynucleotides, sugar modified oliogonucleotides, or carbohydrate modified oligonucleotides (Perbost, et al., (1989), Biochem. Biophys. Res. Commun. 165:742-747), and antisense phosphate-methylated oligodeoxynucleotides (Moody, et al., (1989), Nucleic Acids Res., 17:4769-4782).

The term “gene specific”, as used herein, means an oligonucleotide, oligonucleoside or analog thereof having a sequence that is complementary to a portion of a gene or a portion of a mRNA molecule found in the tissue or cell type targeted by the conjugate. The formation of a sequence-specific duplex between a gene specific pro-drug and the target mRNA will lead to the suppression of expression of the mRNA. The ligands for this delivery system include, but are not restricted to those shown in FIG. 1. Thus, with suitable attachment groups and oligonucleotide sequences, conjugates can be designed which will be effective pharmaceutical compounds for treating diseases and disorders of the liver, such as hepatitis, particularly hepatitis B and cancer of the liver. Additionally, the term “gene specific”, as used herein, means that the pro-drug is an oligonucleoside or oligonucleotide (particularly an oligodeoxynucleoside methylphosphonate or analog thereof) having a sequence that is complementary to a portion of a gene or apportion of a mRNA molecule found in the tissue or cell type targeted by the conjugate. The formation of a sequence-specific duplex between a gene specific pro-drug and the target mRNA will lead to the suppression of expression of the mRNA.

In order to assess the biological effects of this enhanced, cell specific delivery, the integrated hepatitis B viral (HBV) genome was targeted by liver specific neoglycoconjugates in A series of in vitro experiments. HBV is a small enveloped hepadavirus (Tiollais et al., (1985), Nature, 317:489495) that is both a major cause of acute and chronic hepatitis, as well as hepatocellular carcinoma. This virus has a sweeping scope, infecting more than 200 million persons worldwide: The molecular biology of HBV replication has been well characterized and an in vitro model system of hepatoma cells possessing asialoglycoprotein receptors and stably transfected with HBV (Hep G2 2.2.15) has been established (Sells et al., (1987), Proc. Natl. Acad. Sci., 84:1005-1009; Korba and Milman, (1992), Antiviral Res., 19:55-70). Under defined culture conditions, these cells secrete Dane particles into the cell culture media. These particles have been shown to be comprised of a protein coat expressing hepatitis B surface antigen (HBsAG) and a viral DNA core (virion DNA), both of which can be easily assayed in vitro. The corresponding mRNA for these HBV components has been proven to be amenable to modulation by phosphorothioate antisense oligomers (ps-oligomer) (Korba and Gerin, (1995), Antiviral Res., 28:225-242); Goodarzi et al., (1990), J. Gen. Virology, 71:3021-3025); Offensperger et al., (1995), Intervirology, 38:113-119). Recently, enhanced inhibition of HBV replication in transfected liver cells has been demonstrated in vitro by ps-oligomers non-covalently conjugated to DNA carrier systems specific for the asialoglycoprotein receptor (Wu and Wu, (1992), J. Biol. Chem., 267:12436-12439); Madon and Blum, (1996), Hepatology, 24:474481; Yao et al., (1996), Acta. Virologica, 40:35-39).

The cellular uptake and biological efficacy of antisense oligomers directed against integrated HBV is increased significantly by their incorporation into a liver specific neoglycoconjugate via a structurally defined and homogeneous linker system. In this instance, neoglycoconjugate is defined as a conjugate made up of the liver-specific ligand YEE(ahGalNAc)₃ and the desired antisense oligonucleotide covalently joined together by a stable thioether bridge to yield a defined and homogeneous structure. An antisense RNA shall mean an RNA molecule that binds to a complementary mRNA molecule, forming a double-stranded region that inhibits translation of the mRNA. The conjugation of the linker-modified ligand tow pro-drug produces a homogenous, structurally-defined conjugate. Specifically, the linker-ligand entity, such as SMCC-modified YEE(ahGalNAc)₃ is covalently linked to an oligonucleotide to produce a homogenous, structurally-defined neoglycoconjugate. In particular, a carbohydrate-based liver ligand YEE(ahGalNAc)₃ was covalently attached to an oligonucleoside methylphosphonate (ONMP) through a heterobifunctional linker. Such a ligand-linker-prodrug construct directed the oligonucleoside to the liver of mice.

Some examples of HBV-specific oligonucleotides that are directed to various targeted sites are set forth in Table A (Goodarzi, et al., (1990), J. General Virology, 71:3021-3025); Galibert, et al., (1979), Nature, 281:646-650). For example, the core gene encodes the core protein and is essential for HBV DNA replication and is responsible for packaging pre-genomic RNA. The core gene's target is the translational initiation site overlapping polyadenylation site. The surface antigen gene encodes major structural protein of the viral envelope and plays a key role in the pathogenesis of liver damage. The surface antigen gene's target is the translational initiation site. The encapsidation gene encodes for the encapsidation protein and is reponsible for packaging DNA and initiation of HBV DNA synthesis. The encapsidation gene is highly conserved in all HBV strains. Its target is the upper stem/unpaired loop. Other viral-specific oligonucleotides may be synthesized to target specific viral sites as well as gene-specific targets, including cancer-related targets (e.g., raf, ras, and protein kinase).

TABLE A
Hepatitis Viruses

| | |
|---|---|
| Targeted Site | Sequence (5′ to 3′) |

Hepatitis B Virus (HBV)

| | | |
|---|---|---|
| HBx gene | TTGGCAGCACACCCTAGCAGCCATGGA | (SEQ ID NO.:1) |
| HBV surface antigen (S gene) | | |
| Cap site/SPII | GATGACTGTCTCTTA | (SEQ. ID NO.:2) |
| Inside/pre-S2 | AGGAGATTGACGAGA | (SEQ. ID NO.:3) |
| Initiator/gene S | GTTCTCCATGTTCGG | (SEQ. ID NO.:4) |
| Initiator/gene S | TCTCCATGTTCG | (SEQ. ID NO.:5) |
| Inside I/gene S | GAATCCTGATGTAAT | (SEQ. ID NO.:6) |
| Inside II/gene S | AACATGAGGGAAACA | (SEQ. ID NO.:7) |
| PreS 1 open reading frame | | |
| HBV core antigen (C gene) | | |

Hepatitis C Virus (HCV)

| | |
|---|---|
| TFCTCATGGTGCACGGTCTACGA | (SEQ. ID NO.:8) |
| GTTTCGCGACCCAACACTAC | (SEQ. ID NO.:9) |
| CATGATGCACGGTCTACGAGA | (SEQ. ID NO.:10) |
| GCCTTTCGCGACCCAACACT | (SEQ. ID NO.:11) |
| GCCTTTCGCGACCCAAC | (SEQ. ID NO.:12) |
| GCCTTTCGCGACCCAAC | (SEQ. ID NO.:13) |
| GTGCTCATGGTGCACGGTCT | (SEQ. ID NO.:14) |
| GTGCTCATGGTGCACG | (SEQ. ID NO.:15) |
| CTGCTCATGGTGCACGGTCT | (SEQ. ID NO.:16) |

Hepatitis D Virus (HDV)

| | |
|---|---|
| GCGGCAGTCCTCAGT | (SEQ. ID NO.:17) |
| CTCGGCTAGAGGCGG | (SEQ. ID NO.:18) |
| CTCGGACCGGCTCAT | (SEQ. ID NO.:19) |
| TCTTCCGAGGTCCGG | (SEQ. ID NO.:20) |
| ATATCCTATGGAAATCC | (SEQ. ID NO.:21) |
| TGAGTGGAAACCCGC | (SEQ. ID NO.:22) |
| ATTTGCAAGTCAGGATT. | (SEQ. ID NO.:23) |

Using the methods of the invention and other methods known to those in the art, persons of skill in the art will be able to synthesize conjugates of the invention targeting these and other sequences.

Compounds, compositions and methods according to the invention will be useful for treatment of neoplastic and infectious diseases and also include such as variations of carbohydrate-containing ligands, which are directed to the cell surface lectins and specifically for their ligand-binding moieties. In particular, any saccharide or saccharide-modified moieties may be used. A, “physiologically-acceptable carrier” includes any and all solvents, dispersion media, coatings, antibacterial and antifungal-agents, isotonic and absorption-delaying agents, and agents which include ligand-linker-pro-drug (e.g., oligomer) constructs.

A therapeutically-effective dose of a pro-drug of the invention may be administered by intraocular, oral ingestion, inhalation, or intramuscular, intravenous, cutaneous, or subcutaneous injection and may be administered in a pyrogen-free, parenterally-acceptable aqueous solution. A therapeutically effective amount means the total amount of each active component of the pharmaceutical composition of an A-L-P construct or method that is sufficient to show a meaningful patient benefit, i.e., reduction or elimination of a virus or reduction or elimination of the tumor load. When applied to an individual active ingredient, such as delivering a pro-drug to the target, the term refers to that ingredient alone. When applied to a combination, the term refers to combined amounts of the active ingredients that result in the therapeutic effect, whether administered in combination, serially, or simultaneously. The amount of reduction needed for a therapeutic effect will depend upon the molecular and cellular target, disease, and the health status of the patient. For example, in HBV, prefereably at least a 20%, more preferably 50%, and most preferably at least a 70% reduction will be achieved. When a therapeutically effective amount of the invention is administered orally, the conjugate will be in the form of a tablet, capsule, powder, solution, or elixir. The pharmaceutical composition in solution may contain a physiological saline solution, dextrose, or other saccharide solution or ethylene glycol, propylene glycol or polyethylene glycol or any other pharmaceutically acceptable carrier. The amount of conjugate administered in the pharmaceutical composition will depend upon the nature and severity of the condition being treated. Ultimately, the attending physician will decide the dosage and the amount of conjugate of the present invention with which to treat each individual patient, which takes into consideration a variety of factors, such as age, body weight, general health, diet, sex, composition to be administered, route of administration, and severity of the disease being treated. Pharmaceutical compositions containing the A-L-P conjugates of the present invention may be administered to animals including, but not limited to, humans and veterinary animals (e.g., cows, dogs, cats, horses, sheep, and goats), birds and fish.

### EXAMPLES

Synthesis of A-L-P Conjugates

This invention discloses the development of two general conjugation methods, Conjugation Method 1 and Conjugation Method 2 that can be employed to covalently join the oligonucleotide analogs and the neoglycopeptide to yield structurally defined and homogeneous conjugates. Conjugation Method 1 is a three-component reaction that utilizes three chemical species in its conjugation step, the ligand, the functionalized oligonucleotide analog, and the heterobifunctional linker joining the two together. Conjugation Method 2 is a two-component reaction, also referred to as the Quantitative Conjugation Method, that utilizes only two reactants in the conjugation step; the activated ligand and the functionalized oligonucleotide analog. A novel method for radiolabeling of oligonucleotide analogs and their A-L-P conjugates is also disclosed, including those analogs which could not be labeled previously by conventional enzymatic labeling methods. These inventions allowed us to synthesize and radiolabel neoglycopeptide conjugates of virtually every type of oligonucleotide and oligonuceotide analog. An overview of the two conjugation methods and the radiolabeling methods associated with them is given below, is followed by examples illustrating the detailed procedures for using these methods in the synthesis of a variety of A-L-P conjugates.

Conjugation Method 1

This method entails the coupling of a functionalized oligonucleotide analog and the neoglycopeptide using a heterobifunctional cross-linking reagent and is classified as a three-component reaction. The oligonucleotide analog is synthesized in the solid-phase synthesizer. The 5′-end of the oligomer is phosphorylated enzymatically after the solid-phase synthesis to allow further incorporation of a functional group reactive toward the heterobifunctional cross-linking reagent. For oligonucleotide analogs unable to be phosphorylated enzymatically at the 5′-end, such as the methylphosphonate oligomers, an additional nucleotide unit, 2′-O-methyl-nucleotide, is added to the 5′-end via a phosphodiester linkage during the solid-phase synthesis of the oligomer. For example, if a methylphosphonate oligomer T₇ were to be conjugated with the ligand, an oligomer U^{m}pT₇ of the type shown in Table 1, entry 1, is then synthesized by solid-phase method. The oligomer is further modified at its 5′-end with a thiol linker (Table 2, entry 10) post-synthetically and conjugated to YEE(ah-GalNAc)₃ (Table 3, entry 1) with SMCC (Table 4, entry 3), to obtain a conjugate with a linkage identical to the following:

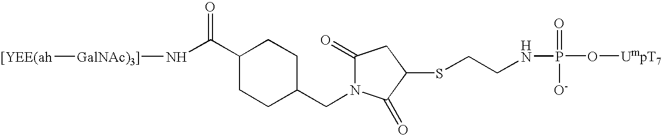

Radiolabeling method associated with Conjugation Method 1. When Conjugation Method 1 is chosen for the synthesis of the A-L-P conjugates, ³²P radiolabeling is easily accomplished at the 5′-end of the oligomer at the enzymatic phosphorylation step by substituting γ-³²P-ATP for the unlabeled ATP. When conjugation is over, the radioactive conjugate can be used immediately in cellular uptake and biodistribution studies.

The detailed procedures for this three-component reaction are further described in the synthesis of a ³²P-labeled A-L-P conjugate named [YEE(ah-GalNAc)₃]-SMCC-AET-pU^{m}pT₇ (10) (Example 1).

Conjugation Method 2

In this method, the neoglycopeptide is modified first at its N-terminal amino group by SMCC to provide the maleimide-activated ligand reactive toward a thiol group (Table 3, entry 6). The SMCC-modified neoglycopeptide is purified to homogeneity before its use in the conjugation reaction. Introduction of a thiol group at the 5′-end of the oligonucleotide analog is achieved conveniently at the solid-phase synthesis stage by incorporating a disulfide linker into the oligomer. Final conjugation is then performed by using purified maleimide-activated neoglycopeptide and purified 5′-thiol-containing oligonucleotide analog. This method of conjugation eliminated all potential side reactions associated with Conjugation Method 1 by using purified activated ligand and oligomer in the conjugation reaction and by careful experimental design and implementation. Conjugation of oligonucleotide analog proceeds quantitatively, allowing easy purification of the final A-L-P conjugates. This reaction scheme is classified as a two-component reaction in which one “half” of the conjugate is modified and then activated for reaction with the other “half”. For example, if the same methylphosphonate oligomer T₇ were to be conjugated with YEE(ah-GalNAc)₃ using SMCC as the heterobifunctional linker, the Conjugation Method 2 would produce a conjugate with a linkage identical to the following:

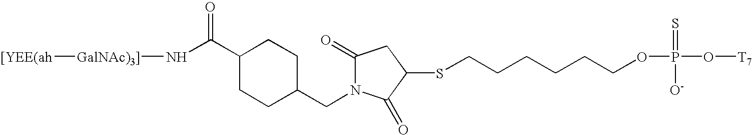

The detailed procedures for this two-component reaction are further described in Example 2 and Example 3.

More examples of the two-component reaction can be realized using similar strategy. For example, the neoglycopeptide can be modified as shown in Table 3, entries 2-5. Activation of the thiol may be accomplished using, for example, 2,2′-dipyridyl disulfide. Reaction of the activated thiol with any of the 3′ or 5′ thiol modified oligomers would provide a disulfide linkage between the oligomer and the neoglycopeptide, as shown below. This scheme provides access to disulfides with varying steric bulk around the sulfur atoms that are not accessible using commercially available crosslinking reagents (Table 4, entries 4-6).

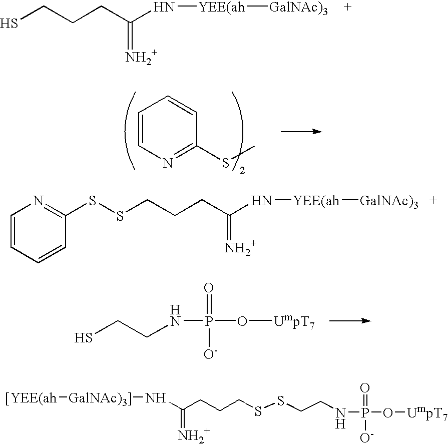

Radiolabeling methods associated with Conjugation Method 2. When Conjugation Method 2 is chosen for the synthesis of the A-L-P conjugates, radiolabeling is performed after the conjugate is synthesized. Radiolabeling of conjugates of certain types of oligonucleotide analogs (e.g., phosphorothioate oligonucleotides) can be accomplished by conventional 3′- or 5′-enzymatic labeling methods, depending oh which end the free hydroxyl group is situated. The radioactive phosphate group is then protected by chemical modification from cellular enzymatic degradation.

For oligonucleotide analogs containing no free hydroxyl group which can participate in enzymatical phosphorylation (e.g., the methylphosphonate oligomers), a method other than enzymatical phosphorylation needed to be developed. For this consideration, we have developed a general method for the incorporation of a stable ³²P-label at the 3′-end of any type of oligonucleotide analog and their A-L-P conjugates, including those which could not be labeled previously by conventional enzymatic labeling methods. The method utilized a combined chemical and enzymatic approach to achieve the labeling and includes the following steps:

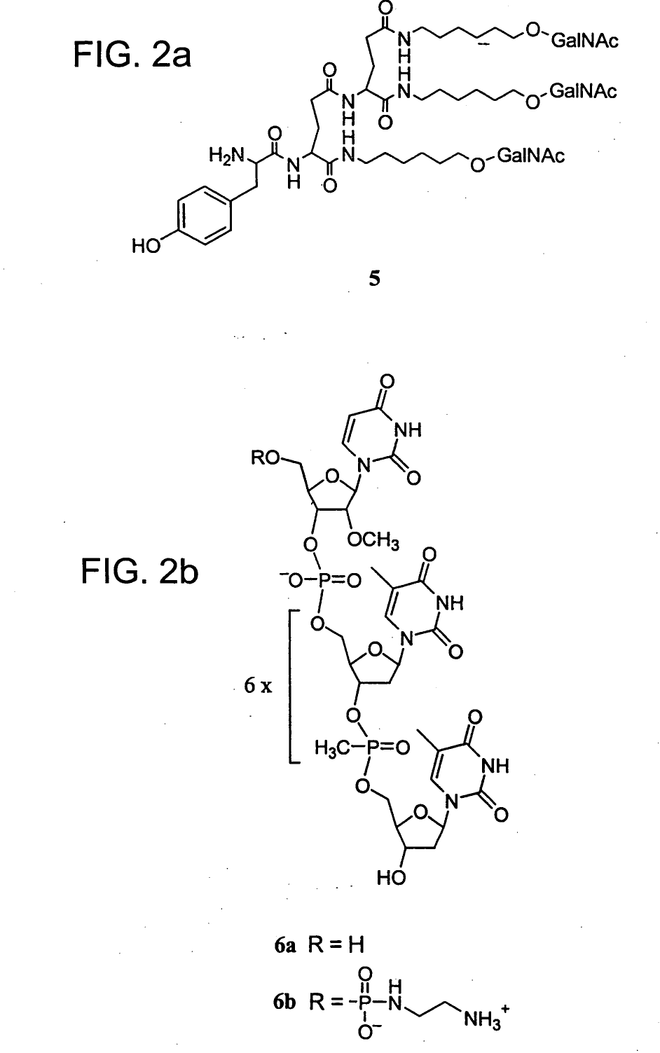

1. Prior to solid-phase synthesis, a hybrid or a chimeric oligomer construct is designed containing three covalently linked segments. The 5′-segment is the disulfide linker. The middle segment is the desired oligonucleotide analog structure. The 3′-end is a phosphorothioate thymidine trinucleotide unit with reversed polarity, 5′-T-3′-3′-TT-5′. This trinucleotide unit is also called the tracer unit, its structure is illustrated in FIG. 2c. Incorporation of this trinucleotide unit introduces a 5′-hydroxyl group at the 3′-end of the oligomer construct, which can be phosphorylated enzymatically.

2. Solid-phase synthesis of the oligomer construct.

3. Ligand conjugation using Conjugation Method 2.

4. ³²P-Labeling of the 3′-end of A-L-P using enzymatic phosphorylation.

5. Chemical modification of the ³²P-labeled phosphate group to protect the label from hydrolysis by cellular enzymes.

FIG. 2d and FIG. 2e illustrated a complete synthesis scheme for the is preparation of a ³²P-labeled A-L-P conjugate 1c, using Conjugation Method 2 and its associated radiolabeling method.

The two methods developed in this invention can be used to synthesize a wide variety of A-L-P conjugates. Examples of oligonucleotide analogs which can be incorporated into the A-L-P conjugates are shown in Table 1. Table 2 lists examples of 3′- and 5′-modification on the oligonucleotide analogs to provide a primary amino group or a thiol group for further reaction. Table 3 shows the neoglycopeptide, which contains an N-terminal amino group, and four methods for modifying the amino group to provide a thiol group, plus an additional method to provide a maleimide group. Finally, Table 4 lists several heterobifunctional cross-linking reagents and a Cathepsin D sensitive oligopeptide, which can be used to link the pro-drug to the ligand. It will be readily apparent that many other reagents are available which would be suitable.

TABLE 1
Oligonucleotide Analogs

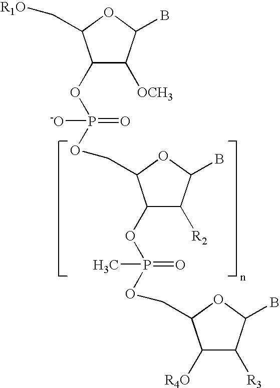

| Entry | R₁ | R₂ | R₃ | R₄ |
|---|---|---|---|---|
| 1 | 5′-conjugate | H | H | H |
| 2 | H | H | H | 3′-conjugate |
| 3 | 5′-conjugate | —OCH₃ | —OCH₃ | H |
| 4 | H | —OCH₃ | —OCH₃ | 3′-conjugate |

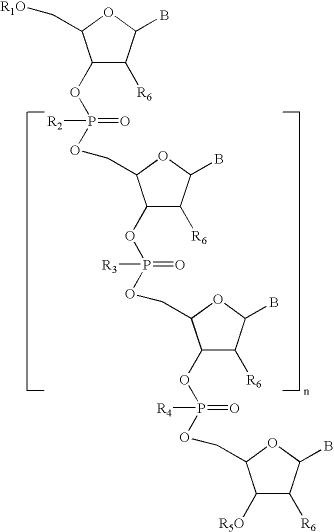

| Entry | R₁ | R₂ | R₃ | R₄ | R₅ |
|---|---|---|---|---|---|
| 5 | 5′-conjugate | O⁻ | CH₃ | O⁻ | H |
| 6 | H | O⁻ | CH₃ | O⁻ | 3′-conjugate |
| 7 | 5′-conjugate | CH₃ | O⁻ | CH₃ | H |
| 8 | H | CH₃ | O⁻ | CH₃ | 3′-conjugate |
| 9 | 5′-conjugate | S⁻ | CH₃ | S⁻ | H |
| 10 | H | S⁻ | CH₃ | S⁻ | 3′-conjugate |
| 11 | 5′-conjugate | CH₃ | S⁻ | CH₃ | H |
| 12 | H | CH₃ | S⁻ | CH₃ | 3′-conjugate |
| 13 | 5′-conjugate | S⁻ | S⁻ | S⁻ | H |
| 14 | H | S⁻ | S⁻ | S⁻ | 3′-conjugate |

B = A, C, G, or T
8 ≦ n ≦ 50
R₆ = H, OH, or OCH₃

TABLE 2
3′ and 5′ modified oligonucleotide analogs for conjugation with
neoglycopeptides.

| Entry | Structure | Functional Group | Reactivity |
|---|---|---|---|
| 1-3 | 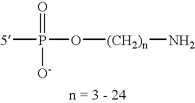 | amino | active esters isothiocyanates isocyanates aldehydes |
| 4 | 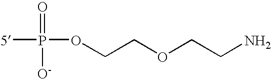 | amino | |
| 5 | 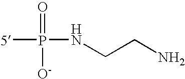 | amino | |
| 6 | 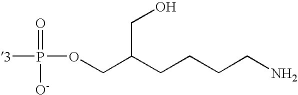 | amino | |
| 7 | 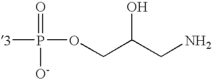 | amino | |
| 8 | 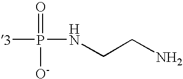 | amino | |
| 9 | 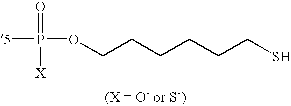 | thiol | 1° halides maleimides activated disulfides |
| 10 | 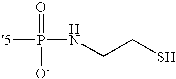 | thiol | |
| 11 | 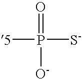 | thiol | |
| 12 | 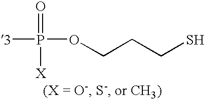 | thiol | |
| 13 | 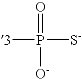 | thiol | |
| 14 | 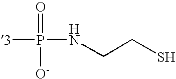 | thiol | |

TABLE 3
Illustrations of functional group modifications to YEE(ah-GalNAc)₃.^{a}

| | | | Reactive | |
|---|---|---|---|---|
| Entry | Modifyiug Reagent | Ligand | Group | Reactivity |
| 1 | none | H₂N-YEE(ah-GalNAc)₃ | amine | active esters |
| | | | | isothiocyanates |
| | | | | isocyanates |
| | | | | aldehydes |
| 2^{b} | 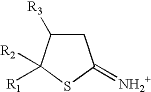 | 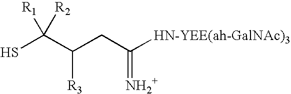 | thiol | 1° halides maleimides activated disulfides |
| 3 | 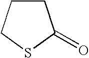 | 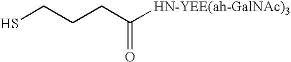 | thiol | |
| 4 | 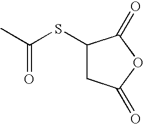 | 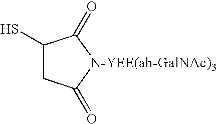 | thiol | |
| 5 | 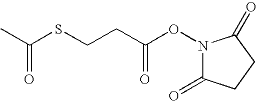 | 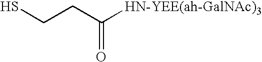 | thiol | |
| 6 | 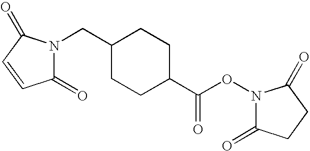 | 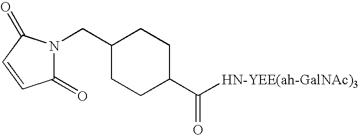 | maleimide | thiol |

^{a}These reagents may be used to modify any of the ligands illustrated in FIG. 1.
^{b}See Goff, D. A.; Carroll, S. F. (1990) Substituted 2-iminothiolanes: reagents for the preparation of disulfide cross-linked conjugates with increased stability Bioconjugate Chem. 1, 381-386.

TABLE 4
Examples of possible combinations of activated oligonucleotide analog, activated
ligand and cross-linking reagent.

| | Reactive Group | | | |
|---|---|---|---|---|
| Entry | Oligomer | Ligand | Cross-Linking Reagent | Linkage |
| 1 | —NH₂ | —NH₂ | 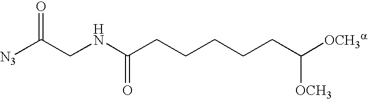 | amide/amine |
| 2 | —SH | —NH₂ | 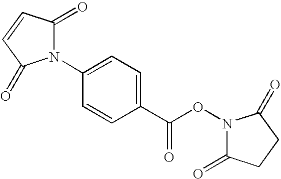 | thioether/amide |
| 3 | —SH | —NH₂ | 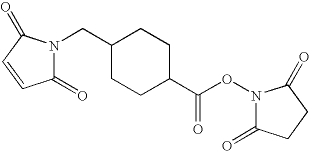 | thioether/amide |
| 4 | —SH | —NH₂ | 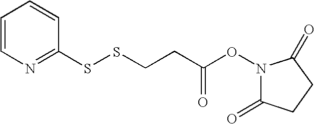 | disulfide/amide |
| 5 | —SH | —NH₂ | 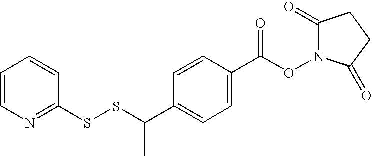 | disulfide/amide |
| 6 | —SH | —NH₂ | 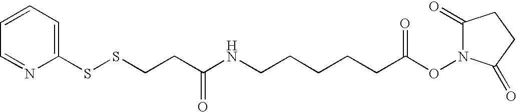 | disulfide/amide |
| 7 | —NH₂ | —SH | see entries 2-6 | amide/thioether |
| | | | | or |
| | | | | amide/disulfide |
| 8 | —NH₂ | —NH₂ | α-citraconyl-K(ε-FMOC)PILFFRL^{a} | amide/amide |
| | | | (cathepsin-D sensitive linker) | |
| 9 | —SH | —SH | requires activation with | disulfide |
| | | | 2,2′-dipyridyl disulfide or | |
| | | | comparable reagent | |

^{a}Reagents shown are not commercially available.

### Example 1

### Synthesis of an A-L-P Conjugate [YEE(ah-GalNAc)₃]-SMCC-AET-pU^{m}pT₇ (10) Using Conjugation Method 1

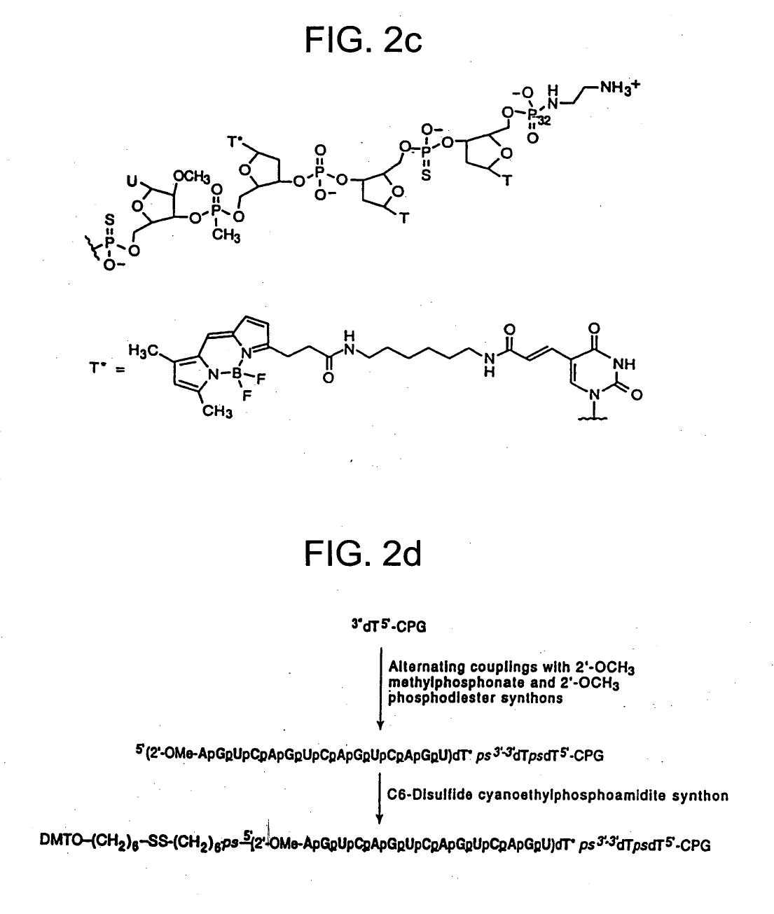

Materials: Methylphosphonamidite synthons were a generous gift from JBL Scientific, Inc., and are commercially available. They can readily synthesized from the nucleoside according to established procedures by an ordinarily skilled practitioner. All other reagents for the automated synthesis of U^{m}pT₇ (FIG. 3) were purchased from Glen Research, Inc. HiTrap Q anion exchange columns were purchased from Pharmacia LKB Biotechnology. Reverse phase high performance liquid chromatography was carried out using Microsorb C-18 column purchased from Rainin Instrument Co., Inc. Cystamine hydrochloride, 1-ethyl-3-[3-(dimethylamino)propyl]carbodiimide (EDAC), 1-methylimidazole, and anhydrous dimethylsulfoxide (DMSO), dithiothreitol (DTT), and Ellmen's reagent were purchased from Aldrich and were used without further purification. Diisopropylethylamime (DIPEA) was purchased from Aldrich and was redistilled from calcium hydride prior to use. N-Hydroxysuccinimidyl-4-(N-methylmaleimido)cyclohexyl carboxylate (SMCC) was purchased from Pierce. Waters SepPak C-18 cartridges were purchased from Millipore Corp. YEE(ah-GalNAc)₃ (FIG. 2a) was synthesized according to Lee et al. (1995, supra) and was stored at 4° C. as an aqueous solution. Adenosine triphosphate (ATP) and [(γ-³²P]-ATP were purchased from P-L Biochemicals, Inc. and Amersham respectively. Polyacrylamide gel electrophoresis (PAGE) was carried out with 20 cm×20 cm×0.75 mm gels which contained 15% polyacrylamide, 0.089 M Tris, 0.089 M boric acid, 0.2 mM EDTA (1×TBE) and 7 M urea. Samples were dissolved in loading buffer containing 90% formamide, 10% 1×TBE, 0.2% bromophenol blue and 0.2% xylene blue.

Synthesis of U^{m}pT₇ (6). The oligodeoxynucleoside methylphosphonate was synthesized on a controlled pore glass support (CPG) using 5′-O-(dimethoxytrityl)-3′-O-methyl-N,N-diispropyl-phosphonamidite thymidine and deprotected according to established methods (Miller, et al., (1991), in Oligonucleotides and Analogues. A Practical Approach (Eckstein, E, Ed.), IRL Press, Oxford, pp. 137-154; Hogrefe, et al., (1993), in Methods on Molecular Biology, Vol. 20: Protocols for Oligonucleotides and Analogs (Aragawal, Ed.), Humana Press, Inc., Totown, pp. 143-164). The final synthon incorporated into the oligomer at its 5′ end was 5′-O-(dimethoxytrityl)-2′-O-methyl-3′-[(2-cyanoethyl)-N,N-diisopropyl]phosphoramidite uridine. The final coupling step positioned a phosphodiester linkage between the terminal 5′ nucleoside and the adjacent nucleoside, which permitted phosphorylation of the 5′ terminal hydroxyl group with bacteriophage T4 polynucleotide kinase and ensured reasonable stability of the phosphodiester due to the presence of the 2′-O-methyl group. The crude 8-mer was purified by HiTrap Q anion exchange chromatography (load with buffer containing <25% acetonitrile; elute with 0.1 M sodium phosphate, pH 5.8) and preparative reverse phase chromatography (Microsorb C-18) using a linear gradient (Solvent A: 50 mM sodium phosphate, pH 5.8, 2% acetonitrile; Solvent B: 50 mM sodium phosphate, pH 5.8, 50% acetonitrile; gradient: 0-60% B in 30 min). The oligomer thus purified was ca 97% pure by analytical HPLC, only contaminated by a small amount of the n-1 species.

Synthesis of [5′-³²P]-5′-O-[(N-2-thioethyl)phosphoramidate]-U^{m}pT₇ (9) (FIG. 3). The purified oligomer (168 nmol), ATP (160 nmol), H₂O (75 μL), 10×PNK buffer (5 mM DTT, 50 mM Tris(HCl, 5 mM MgCl₂, pH 7.6; 10 μL), [γ-³²P]-ATP (3000 Ci/mmol, 100 μCi, 10 μL), and PNK (150 U in 5 μL) were combined and incubated at 37° C. for 16 hours and evaporated to dryness. The residue was redissolved in 0.2 M 1-methylimidazole, pH 7.0 (100 μL) and 1.0 M cystamine hydrochloride, pH 7.2, containing 0.3 M EDAC (100 μL) and heated at 50° C. for 2 hours (Chu, et al., (1983), Nucleic Acid Res., 11:6513-6529; Chu et al., (1988), Nucleic Acid Res., 16:3671-3691). The excess reagents were removed by SepPak (loaded with 50 mM sodium phosphate, pH 5.8, 5% acetonitrile; washed with 5% acetonitrile in water; eluted with 50% acetonitrile in water). The solvent was evaporated in vacuo and crude cystamine adduct redissolved in 10 mM phosphate containing 50 mM DTT (200 mL) and heated to 37° C. for 1 hour. The buffer salts and the excess reductant were removed from the reaction mixture as before and the crude product was dried in vacuo. The title compound 9, produced in 57% yield from 6, was used in the next step without further purification.

Synthesis of [5′-³²P]-[YEE(ah-GalNAc)₃]-SMCC-AET-pU^{m}pT₇ (10). The neoglycopeptide 5 (336 nmol) (FIG. 2a) was dissolved in anhydrous DMSO (4 mL) and treated with DIPEA (336 nmol) and SMCC (336 nmol). The reaction was allowed to stand at room temperature for 4 hours, then added to the freshly prepared thiol 9 (FIG. 3). The reaction mixture was degassed and allowed to slowly concentrate under vacuum at room temperature. The crude was dissolved in formamide loading buffer (100 μL, purified by PAGE (4 V/cm, 1.5 hour), and recovered by the crush and soak method (50% acetonitrile in water). The overall yield of pure 10 was 25%. 10 produced [5′-³²P]-phosphorylated 6 upon treatment with 0.1 N HCl (37° C., 1 hour) due to hydrolysis of the P—N bond; however, 6 was unreactive towards DTT (50 mM, pH 8, 37° C., 1 hour), 3-maleimidopropionic acid (50 mM, pH 8, 37° C., 1 hour), Ellman's reagent (50 mM, pH 8, 37° C., 1 hour) and BAP (70 U, 65° C., 1 hour). Sequential treatment of 10 with 0.1 N HCl and BAP resulted in complete loss of [³²P]-label as anticipated. Stoichiometric analysis of an unlabeled sample of 10 prepared in an identical manner showed it to contain 3 moles of N-acetylgalactosamine for each mole of conjugate, consistent with the proposed structure. (The molar absorptivity of U^{m}pT₇ was calculated to be 59,750 L/mol-cm by taking the sum of the molar absorptivity values for each of the nucleosides contained in the structure. This value was in excellent agreement with the number of moles of GalNAc residues found contained in the conjugate). Pneumatically assisted electrospray mass spectrometry produced a parent ion (negative ion mode) at M/Z 4080 (calculated mass 4080.7).

Discussion of Method 1.

Synthesis and purification of YEE(ah-GalNAc)₃ and pU^{m}pT₇ were carried out according to established procedures (Lee, (1987), supra; Miller, (1991), supra). In order to form a covalent link between YEE(ah-GalNAc)₃ and U^{m}pT₇, the 5′-end of U^{m}pT₇ was modified using the method Orgel (Chu, (1983 & 1988), supra). This introduced a disulfide into the oligo-MP, which in turn could be reduced with DTT to give a 5′-thiol. The neoglycopeptide (FIG. 2a) was modified in a complementary fashion using the heterobifunctional cross-linking reagent, SMCC, capable of combining specifically with the N-terminal amino group of YEE(ah-GalNAc)₃. Coupling of the maleimido group introduced by SMCC and the 5′-thiol of the modified oligo-MP resulted in linkage of the oligo-MP and neoglycopeptide via a metabolically stable is thioether (FIG. 3).

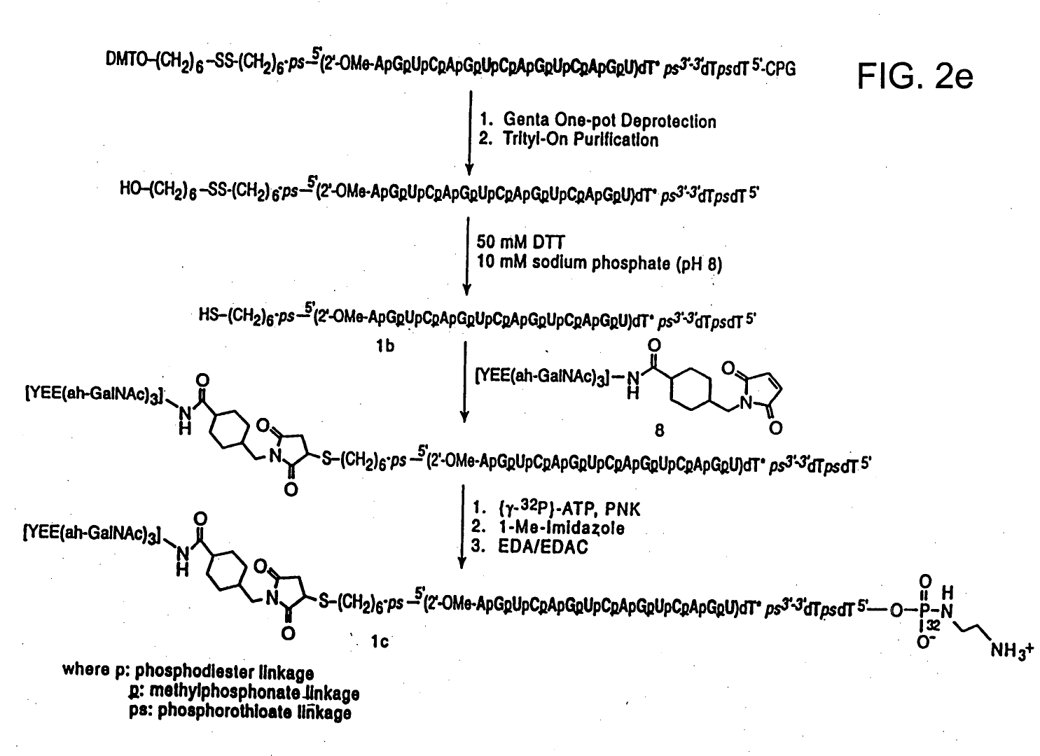

To begin the synthesis, U^{m}pT₇ was phosphorylated using PNK and 0.95 equivalent of [³²P]-ATP. Successful 5′-phosphorylation was confirmed by an increase in the electrophoretic mobility of the product compared to the parent oligo-MP owing to the increased negative charge from −1 to −3 upon addition of a 5′-phosphate and incorporation of ³²P into the structure (FIG. 4, band A). Formulation of the end-labeling reaction in this way ensured that about 90% of the ATP was consumed, allowing efficient use of the [³²P]-ATP to radioactively label the conjugate. Modification of the 5′-phosphate was accomplished in two steps. The 5′-end-labeled oligo-MP was incubated at 50° C. with 0.5 M cystamine hydrochloride in a buffer containing 0.1 M 1-methyllimidazole at pH 7.2 in the presence of 0.15 M EDAC to give the 5′-cystamine phosphoramidate in 65% yield. PAGE analysis of the reaction mixture showed the product to migrate significantly slower than the 5′-end-labeled oligo-MP. This observation is consistent with the change from −3 to −1 due to the loss of a single oxyanion on the 5′-phosphate upon formation of the P—N bond and neutralization of a second negative charge by the positively charged protonated primary amine present on the terminua of the cystamine group (FIG. 4; compare bands A and B). Up to 35% of thymidine-modified oligo-MP was produced during this reaction (FIG. 4, band C), and despite attempts to modify the reaction conditions (e.g., lowering the termperature and reducing the concentration of EDAC), its production could not be eliminated without concomitant reduction in yield of the desired cystamine adduct. This side product presumably arises due to reaction of EDAC with N-3 of thymidine to form a thymidine-EDAC adduct (Chu, supra; Gilham, supra). Reduction of the disulfide with 50 mM DTT at pH 8 was quantitative and was accompanied by mobility shift to a faster migrating species due to the loss of the positively charged protonated primary amino group (FIG. 4; compare bands B and C). In a separate reaction, YEE(ah-GalNAc)₃ was combined with 1 equiv each of SMCC and DIPEA in anhydrous DMSO and incubated at room temperature. Combination of this reaction mixture with thiol 9 could be carried out without complete consumption of SMCC by YEE(ah-GalNAc)₃ since the reactive groups present on 5, 7, and 9 combined regiospecifically, thereby yielding a structurally defined and homogeneous conjugate. As anticipated, the addition of the modified neoglycopeptide to the 5′-end of the activated oligo-mp was accompanied by a substantial slowing of its mobility by PAGE since the mass of the conjugate 10 is significantly larger than that of the parent oligo-MP (FIG. 4, band F). Following this scheme, 9 was completely converted to 10 when 2 equiv (based on starting oligo-mp 6 of the neoglycopeptide YEE(ah-GalNAc)₃ was used. The overall yield of the conjugate 10 was 24% (average of three syntheses) based on oligo-mp 6. The homogeneity of 10 was confirmed by the detection of a single parent ion (negative ion mode) by electrospray mass spectrometry.

### Example 2

### Synthesis of A-L-P Conjugate 1c Using Conjugation Method

This example describes the detailed procedures for using the Conjugation Method 2 in the synthesis of A-L-P conjugates from a novel type of oligonucleotide analogs, the 2′-O-methyl ribose alternating methyl-phosphonate-phosphodiester backbone. Table 5 listed three oligomers of this type (oligomers 1-3), and their A-L-P conjugates formed with the liver ligand YEE(ah-GalNAc)₃ (conjugate 1c, 2c, 3c). The following describes procedures for the synthesis of conjugate 1c. The other two conjugates were synthesized similarly.

The procedures for using Conjugation Method 2 in the synthesis of 1c involves the following steps: 1) Synthesis of SMCC-YEE(ah-GalNAc)₃ (8); 2) Designing of Oligomer Construct 1b; 3) Solid-phase synthesis of oligomer construct 1b; and 4) Conjugation of 1b with SMCC-YEE(ah-GalNAc)₃-synthesis of 1c. 5) ³²P Radiolabeling of conjugate 1c.

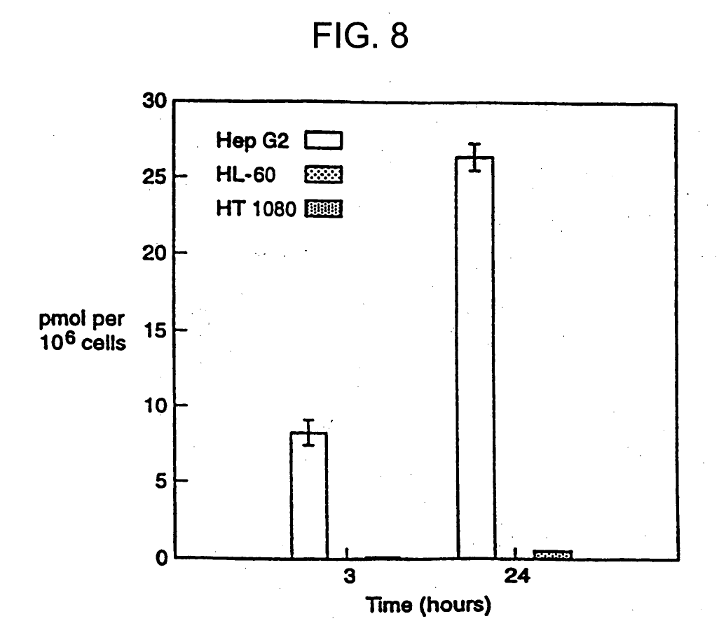

TABLE 5
Oligonucleotide Alternating Methylphosphonate Analogs.
Sequence

| | |
|---|---|
| 1 (n = 7) | ApGpUpCpApGpUpCpApGpUpCpApGpU |
| 2 (n = 7) | GpUpUpCpUpCpCpApUpGpUpUpCpApG |
| 3 (n = 10) | UpUpUpApUpApApGpGpGpUpCpGpApUpGpUpCpCpApU |

where
p: phosphodiester linkage
p: methylphosphonate linkage
ps: phosphorothioate linkage

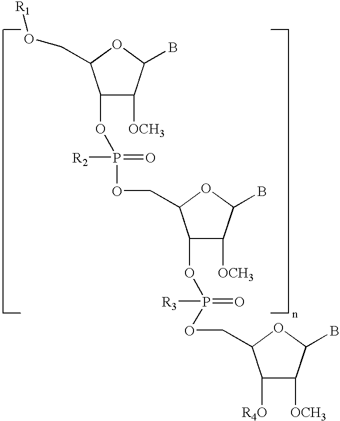

| Oligonucleotide | R₁ | R₂ | R₃ | R₄ |
|---|---|---|---|---|
| a | H | O⁻ | CH₃ | 3′-conjugate |
| b | C6-thiol-ps | O⁻ | CH₃ | 3′-conjugate |
| C | 5′-conjugate | O⁻ | CH₃ | 3′-conjugate |
| d | Ligand-SMCC-AET | O⁻ | CH₃ | H |
| e | EDA | O⁻ | CH₃ | H |

where
Ligand: YEE(ah-GalNAc)₃
5′-conjugate: YEE(ah-GalNAc)₃-SMCC-S(CH₂) ₆-ps linkage (FIG. 3)
3′-conjugate: Tracer Unit (FIG. 9)
EDA: ethylenediamine

In Table 5, oligomers 1-3 can be linked with substituent groups indicated as oligonucleotides a-e at the bottom of Table using the synthesis methods described herein below to form further examples of compounds of the invention. For example, 1b consists of sequence 1 with substituents according to the invention of C6-thiol-ps, O⁻, CH₃, and 3′-conjugate (the structure of which is shown in FIG. 2c). Compounds of the structures indicated by 1b (FIG. 2e) and 1c were synthesized according to the scheme shown in FIGS. 2d and 2e, as set forth in detail in Example 2. It will be clear that with suitable substitution in starting material and changes in the synthesis the other combinations can be similarly synthesized.

Synthesis and Purification of SMCC-YEE(ah-GalNAc)₃ (8). About 1-2 μmole of YEE(ah-GalNAc)₃ was dried into a 1 mL glass Reacti-vial. To this solution, anhydrous DMSO (250 μL) and anhydrous DIPEA (3 μL) was added, then treated with 150 μL of a solution containing vacuum-dried SMCC (6 mg) in anhydrous DMSO. The mixture was vortexed briefly and left standing at room temperature. The progress of reaction was monitored by reversed-phase HPLC analysis in 30 minute intervals. HPLC methods: Microsorb C18 250×4.6 mm. 0-5 min, 0-40% B; 5-20 min, 40-100% B. A: 2% CAN in 50 mM sodium phosphate pH 5.8; B: 50% CAN in 50 mM sodium phosphate pH 5.8. Flow rate 1 mL/min. Detection: A280 nm. The results of HPLC analysis indicated complete-conversion of the starting YEE(ah-GalNAc)₃ (elution time: 7.3 minutes) to the desired product SMCC-YEE(ah-GalNAc)₃ (elution time: 9.8 minutes) in 2 hours. The reaction mixture was then diluted to 10 mL with 50 mM sodium phosphate (pH 5.8) containing 2% CH₃CN and was loaded onto a Sep-Pak cartridge. The cartridge was washed with 10 mL of 50 mM (pH sodium phosphate 5.8) containing 2% CH₃CN and the product was eluted with 10 mL of 25% CH₃CN/H₂O. The product was concentrated under reduced pressure in a Speed-vac and was further purified on a semi-preparative reversed-phase C18 column. HPLC methods: Microsorb C18 250×7.5 mm. 0-60 min, 20-60% B. A: 2% CAN in 50 mM sodium phosphate pH 5.8; B: 50% CAN in 50 mM sodium phosphate pH 5.8. Flow rate 2 mL/min. Detection: A280 nm. Fractions containing pure SMCC-YEE(ahGalNAc)₃ were pooled and desalted on a Sep-Pak. Final yield of product: 1.89(O.D. 276 or 1.35 μmole). UV (25% CAN): 305 (br), 282 (sh), and 276 nm. Relative intensities: ε305:ε282:ε276=1:2.7:3.0. MS(electrospray, positive ion mode): 1565 (M+H)⁺. Analytical reversed phase HPLC: a single peak at 9.8 min. (HPLC conditions: Microsorb C18 250×4.6 mm. 0-5 min, 0-40% B; 5-20 min, 40-100% B. A: 2% CAN in 50 mM sodium phosphate pH 5.8; B: 50% ACN in 50 mM sodium phosphate pH 5.8. Flow rate 1 mL/min. Detection: A280 nm.).

Designing of Oligomer Construct 1b (Table 5). The following description illustrates how to design an oligomer construct for the purpose of conjugating an oligonucleotide analog 1 with YEE(ah-GalNAc)₃ and to allow ³²P-labeling of the A-L-P conjugate formed. Oligomer 1 belongs to a novel type of oligomers containing 2′-O-methylribose with alternating phosphodiester and methylphosphonate internucleotide linkages, its sequence is illustrated in Table 5. Oligomer 1b (Table 5) is the construct designed to allow oligomer 1 to be conjugated with the ligand and for the conjugate to be ³²P-labeled. Oligomer 1b comprises of three portions. The middle portion contains the exact structure of oligomer 1, i.e., the P portion of A-L-P. The 5′-portion contains a C6-thiol linker, which attaches to the 5′-A of oligomer 1 through a phosphorothioate linkage. This thiol group will be used for conjugation with the SMCC-modified ligand, i.e., the A portion of A-L-P. The 3′ portion is a tracer unit covalently attached to the 3′-U of oligomer 1 through a methylphosphonate linkage. The tracer unit is a phosphorothioate thymidine trinucleotide unit with reversed polarity, 5′-T-3′-3′-TT-5′, its structure is illustrated in FIG. 9 or 2c. Without this trinucleotide unit, the final conjugate would have, at its 3′-end, a 2′-O-methyl-uridine with a 5′-methylphosphonate linkage. Radiolabeling this 3′-end would become impossible by conventional enzymatic methods. Incorporation of this trinucleotide unit introduces a 5′-hydroxyl group at the 3′-end of the oligomer construct, which can be phosphorylated enzymatically to allow ³²P-labeling of the conjugate.

The 3′-tracer unit is needed only when it is desired to label the final conjugate with ³²P For applications that do not require a radiolabeled conjugate, this tracer unit is omitted, and the oligomer construct comprises only the thiol linker and the P portions.

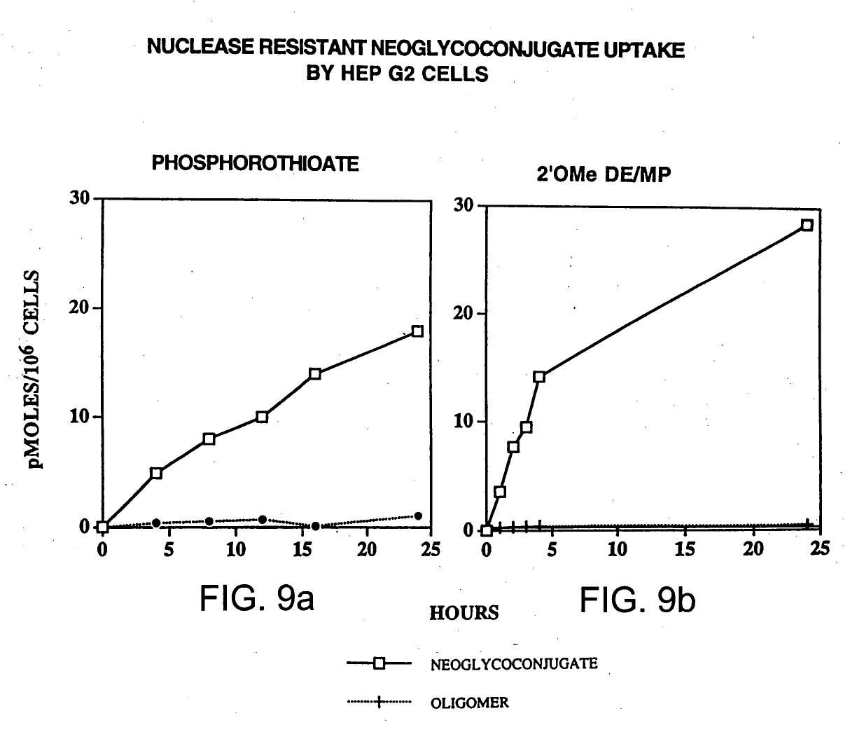

Solid-phase synthesis of oligomer construct 1b. The modified oligomer 1b was synthesized on a solid-phase DNA synthesizer, using corresponding phosphoramidites and methyl-phosphonamidites from a commercial source (Glen Research). The tracer was assembled (FIG. 10) using phosphorothioate chemistry on dT-5′-Lcaa-CPG support by coupling to the support the 3′-DMT-dT-5′-CE phosphoramidite, followed by 5′-DMT-5-[N(−)trifluoroacetyl)hexyl-3-acrylimide]-2′-deoxyuridine 3′-[(2-cyanoethyl)-(N,N′-diisopropyl)]phosphoramidite (the CPG and synthons were commercially available from Glen Research), Sequence corresponding to oligomer 1 was then assembled onto the tracer-containing CPG by sequencially coupling of the 2′-O-methyl methylphosphonamidite, synthons and 2′-O-methyl-cyanoethylphosphoramidite synthons. The 5′-disulfide linker was then introduced into the oligomer by coupling, a C6-disulfide cyanoethyl-phosphoramidite synthon (Glen Research) using phosphorothioate chemistry at the final coupling step of the solid-phase synthesis. When necessary, the Beaucage reagent (Glen Research) was substituted for the low moisture oxidizer to effect sulfurization of the phosphite to give the phosphorothioate according to standard established procedures. The oligomer was synthesized without the removal of the 5′-DMT group. The oligomer was deprotected under Genta one-pot method (Hogrefe, R. I., Vaghefi, M. M., Reynolds, M. A., Young, K. M. and Arnold, L. J. (1993) Nucl. Acids Res., 21, 2031-2038) and were purified by trityl-on procedures. The disulfide-containing oligomer was finally purified using a semi-preparative reversed-phase C18 column. HPLC conditions: Microsorb C18 250×7.5 mm. 0-50 min, 20-60% B. A: 2% CAN in 50 mM sodium phsphate pH 7; B: 50% CAN in 50 mM sodium phosphate pH7. Flow rate 2 mL/min. Detection: A254 nm.

The reduction of the disulfide moiety to the thiol was effected by the treatment of the 5′-disulfide-containing oligomer with DTT. Thus, a 2.5 O.D. 260 (˜16 nmole) disulfide oligomer was dissolved in 400 μL of freshly prepared and degassed 50 mM DTT solution in 10 mM sodium phosphate, pH 8. The mixture was incubated at 37° C. for 2 hours. Quantitative reduction was confirmed by reversed-phase HPLC analysis, which shows that the thiol oligomers elute faster than the parent disulfide oligomers. The thiol oligomer was then purified on a Sephadex G-25 column (10×300 mm) to remove DTT and salts. Column packing and sample elution were effected by the use of degassed 20% ethanol-water. The G-25 fraction containing the pure thiol oligomer was used immediately in the next reaction to minimize unwanted oxidation. Synthesis of YEE(ah-GalNAc)₃-containing oligomer 1c.

The G-25 fraction containing 1.8 O.D. 260 (12 nmoles) pure thiol oligomer (1b) was mixed with SMCC-YEE(ah-GalNAc)₃ (50 nmole) immediately after it was collected. The mixture was concentrated to dryness in a speed-vac. The residue was dissolved in 100-μL of degassed 50% CH₃CN containing 0.1 M sodium phosphate, pH 7. The solution was further degassed in a speed-vac by applying vacuum for about 5 minutes. The solution was then capped tight and incubated at room temperature overnight to allow conjugation to complete. Alternatively, conjugation can be performed by mixing the freshly-collected thiol-oligomer G25 fraction with a solution of SMCC-YEE(ah-GalNAc)₃ in 50% CH₃CN containing 0.1 M sodium phosphate, pH 7. The solution was immediately placed in a speed-vac and concentrated to about 1 ml. The solution was then capped tight and incubated at room temperature overnight to allow conjugation to complete. Both procedures have been found to give quantitative conjugation of the thiol-containing oligomers.

To determine the yield of the conjugation reaction, about 0.5 μL portion of the reaction was dried and phosphorylated using [γ⁻³²P]-ATP and PNK and analyzed by 20% denaturing PAGE. The mobility of the conjugate was compared with that of the unconjugated oligomer in the same gel. Unlabeled conjugate can also be analyzed in similar fashion by UV shadowing. The PAGE results indicated quantitative conjugation of the thiol oligomer with the neoglycopeptide. The conjugate was confirmed by its significant gel mobility shift upon chymotrypsin digestion and its inability to shift upon DTT treatment. The conjugate was finally purified by a Sephadex G25 column, eluting with 20% ethanol. The purified 1c can be used directly in bioefficacy experiments and other experiments which do not require a radiolabeled conjugate.

³²P Radiolabeling of Conjugate 1c. In order to use 1c in cellular uptake and biodistribution experiments, 1c was labeled with ³²P by the use of γ-³²P ATP and PNK according to conventional 5′-enzymatic radiolabeling procedures. The purified conjugate 1c (10 nmol), ATP (10 nmol), H₂O (70 μL), 1O× PNK buffer (5 mM DOT, 50 mM Tris(HCl, 5 mM MgCl₂, pH 7.6; 10 μL), [γ-³²P]-ATP (3000 Ci/mmol, 150 μCi, 15 μL), and PNK (300 U in 10 μL) were combined and incubated at 37° C. for 16 hours. Incorporation of ³²P into the conjugate was assayed by 15% PAGE and autoradiography. To the labeling solution were then added 0.2 M 1-methylimidazole, pH 7.0 (100 μL) and 1.0 M ethylenediamine hydrochloride, pH 7.2, containing 0.3 M EDAC (100 μL). The solution was then incubated at 50° C. for 2 hours (Chu, et al., (1983), Nucleic Acid Res., 11:6513-6529; Chu et al., (1988), Nucleic Acid Res., 16:3671-3691). The excess reagents were removed by a NAP-25 column eluted with 20% aqueous ethanol. Fractions containing pure ³²P-labeled conjugate were then assayed by UV absorbance measurement and scientilation counting. The specific activity was calculated to be around 5-8 Ci/mmole. The purified ³²P-labeled conjugate was assayed again by 15% PAGE and autoradiography to be free of any low molecular weight ³²P contaminates.

### Example 3

### Synthesis of A-L-P Conjugates-NG1 to NG5 Using Conjugation Method 2

The conjugation Method 2, as described in Example 2 in this invention, is a general method that can be used to form A-L-P conjugates of any oligonucleotide analogs. The following description is another example to use the conjugation Method 2 for the synthesis of a different type of A-L-P conjugates. In this example, the oligonucleotide analogs to be conjugated belong to the type of oligodeoxyribonucleoside phosphorothioates, one of the major types of analogs used in current antisense drug development worldwide. Because oligodeoxyribonucleoside phosphorothioates are easily labeled at the 3′-end by classical 3′-enzymatic labeling procedures, the 3′-tracer unit, as was used in example 2, is not needed here for the conjugates to be radiolabeled. Therefore, the oligomer constructs to be designed contain only two portions, the phosphorothioate oligomer portion and the 5′-disulfide linker portion.

These 5′-disulfide-containing phosphorothioate oligonucleotides were synthesized via automated phosphorothioate oligonucleotide synthesis method on an ABI 392 DNA/RNA synthesizer, using the normal 3′-CPG supports, 5′-DMT-nucleoside 3′-• -cyanoethyl phosphoramidites, and C6-thiol linker • -cyanoethyl phosphoramidite. (All of these reagents are commercially available from Glen Research, Sterling, Va.) The oligomers were synthesized without the removal of the 5′-DMT group. The oligomers were deprotected with concentrated ammonium hydroxide for 16-20 hours at 55° C. The trityl-containing oligomers were then purified by preparative reversed phase HPLC with a Microsorb C-18 column using a linear gradient of acetonitrile in 50 mM sodium phosphate pH 7.5. The purified trityl-containing oligomers were detritylated by 0.5% TFA on Sep-Pak (Waters, Milford, Mass.). All oligonucleotides were desalted on Sep-Pak columns before subsequent experimental use.

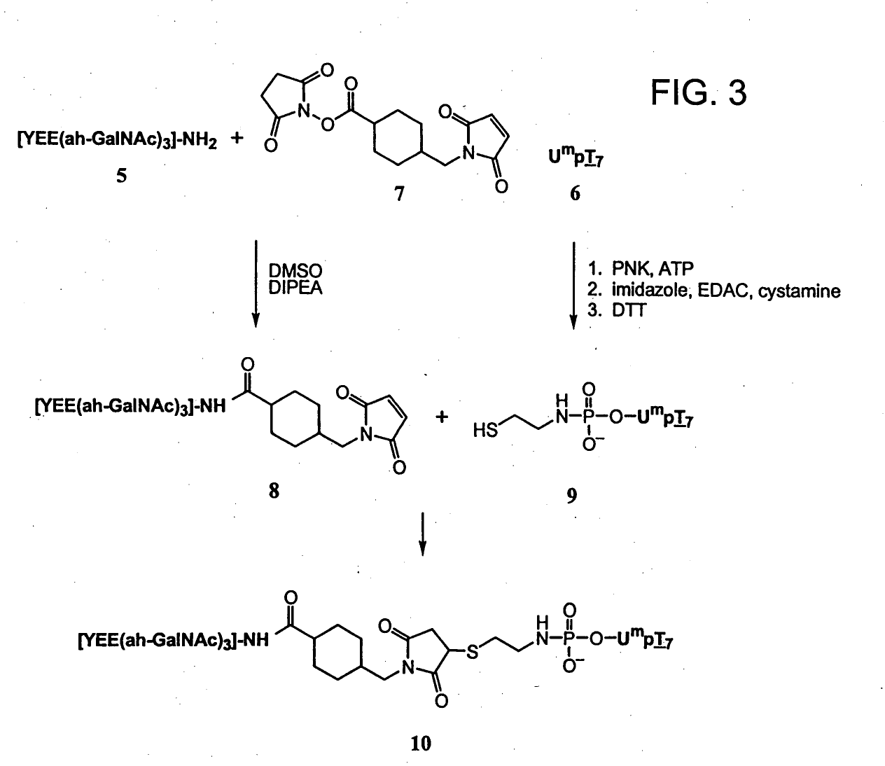

The purified disulfide-containing oligomers were then used in conjugation with SMCC-YEE(ah-GalNAc)₃ similarly as described in Example 2. Most conjugation reactions were performed by using 1.5-2 equivalents of SMCC-YEE(ah-GalNAc)₃ to the thiol oligomers. These resulted in quantitative conjugation of the oligomers in all of the reactions performed. Excess ligand and buffer salts were easily removed by a G-25 column, eluting with 20% ethanol, to give highly pure conjugates. Conjugation reactions were also performed using excess amount of thiol oligomers instead, e.g., 1.5 equivalent of the thiol oligomers to the ligand. In these cases, all of the ligands were consumed in the reactions and the remaining excess amount of thiol-oligomers were removed by preparative reversed phase high pressure liquid chromatagraphy (HPLC). Following are the sequences of five oligodeoxyribonucleoside phosphorothioate A-L-P conjugates synthesized by the above method (FIG. 5). NG1: YEE(ahGalNAc)₃-SMCC-5′GTTCTCCATGTTCAG3′, which targeted the HBV sa-gene, NG2: YEE(ahGalNAc)₃-SMCC-5′TTTATAAGGGTCGATGTCCAT3′, which targeted the HBV c-gene, NG3: YEE(ahGalNAc)₃-SMCC-5′AAAGCCACCCAAGGCA3′, which targeted the HBV e-site, and the random controls, NG4: YEE(ahGalNAc)₃-SMCC-5TGAGCTATGCACATTCAGATT T3′, and NG5: YEE(ahGalNAc)₃′-SMCC-5′TCCAATTAGATCAG3′.

Several methods have been employed to determine the yields of oligomer conjugation and the purity of the conjugates. 1) PAGE analysis of ³⁵S-labeled conjugation reaction mixture. About 0.5 μL portion of the reaction was dried and labeled at 3′-end with [35 S]-ATP-S (see 3′-labeling procedure described below) and analyzed by 20% denaturing; 2) PAGE analysis of unlabeled conjugation reaction mixture visualized by UV shadowing; and 3) HPLC analysis of unlabeled conjugation reaction mixtures. All samples were treated with DTT before subjected to the above three analyses. Unconjugated thiol-oligomers were treated and analyzed in parallel for comparison purposes. It was found in the PAGE analyses that the conjugates all showed a significantly slower mobility on the gel than the corresponding thiol oligomers, and that all of the thiol oligomers were fully converted to the corresponding conjugates. In reversed phase HPLC analysis, all conjugates showed a single peak and their retention were about 2-3 min longer than those of the unconjugated thiol-oligomers.

Several methods have been employed to characterize the final conjugates. The conjugates were subjected to the following treatment and then analyzed by both PAGE and HPLC analysis: 1) Chymotrypsin digestion; 2) NAGA digestion; and 3) DTT treatment. Chymotrypsin digestion generated an oligomer species, which migrated faster on the gel than both the conjugate and the thiol oligomer, confirming the presence of tri-peptide structure in the conjugates. HPLC analysis also indicated change in retention times upon the digestion. The presence of the sugar moiety was confirmed by digestion with NAGA which generated an oligomer species migrating only a little bit faster than the conjugate on the gel but this species showed a significant longer retention in HPLC than the conjugate. The DTT treatment did not result in any change to the conjugate structure based bn the gel and HPLC analysis, indicating that disulfide is absent in the structure and that all thiol groups have participated in the conjugation reaction. The structures of the conjugates were also confirmed by pneumatically assisted electrospray mass spectrometry.

³⁵S Radiolabeling of Conjugate NG1. A representative (NG1) conjugate was labeled with ³⁵S and assayed for cellular uptake in both Hep G2 and Hep G2 2.2.15 cells. The ³⁵S radiolabel was incorporated at the 3′-terminal using the combined action of terminal deoxynucleotidyltransferase (Life Technologies, Grand Island, N.Y.) and [³⁵S] dATP • S (>1000 Ci/mmole) (Amersham Biotech, Piscataway, N.J.). All ³⁵S labeled oligomers were purified by either Sephadex G25 columns or Sep-Pak cartridges before used in cellular experiments.

Comparison of Conjugation Method 1 and Conjugation Method 2.

Conjugation Method 1 was a general conjugation method developed earlier in this invention. It has been used successfully in the synthesis of A-L-P conjugates from oligonucleoside methylphosphonates (e.g., conjugate 10) and their analogs containing alternating phosphodiester-methylphosphonate backbone (e.g., conjugate 1d, Table 5). It provided a method for the construction of these chemically-defined and structurally homogeneous A-L-P conjugates and played an important role in this invention. However, this method needed several improvements: 1) Side reactions need to be minimized. These side reactions include the EDAC-adduct formation, the conjugation of unreacted SMCC to the thiol oligomer, and the conjugation of hydrolyzed SMCC to the thiol oligomer. These side reactions decreased the yield of product formation and produced a mixture which required the use of PAGE as one of the is purification methods, which further decreased the overall yield to around 25%. 2) The chemistry needed to be refined in order to use this method to synthesize A-L-P conjugates of oligomers of other backbones, e.g., the phosphorothioates. The use of EDAC and cystamine in the modification of the 5′-phosphate is not suitable for this type of oligomer. 3) ³²P-labeling can not be performed after the ligand conjugation is finished. Due to the relatively short half-life of this label, the whole conjugation procedure must to be repeated whenever fresh ³²P-labeled conjugate is needed.

The conjugation Method 2 offers significant improvement over Method 1 in its quantitative conjugation of oligomers with the ligand, universal compatibility with all types of oligomer backbones, easy purification of the conjugates, and flexibility in radiolabeling of the conjugates. These improvements were achieved through the implementation of the following procedures unique in Method 2:

1) By incorporating the thiol linker into the oligomer construct during the solid-phase synthesis, post-synthesis modification of the oligonucleotide analogs is avoided. This eliminated the EDAC-adduct formation as found in method 1. This also made it possible to synthesize conjugates of oligonucleotides of certain backbone types susceptible to EDAC modification, e.g., the phosphorothioate oligonucleotides. It is therefore possible to synthesize A-L-P conjugates of all types of oligonucleotide analogs to which a thiol linker can be incorporated into their structures.

2) By synthesizing the activated ligand scaffold, YEE(ah-GalNAc)₃-SMCC, and purifying it to homogeneity before its use in the conjugation reaction, unwanted conjugates, such as the conjugates with unreacted SMCC and hydrolyzed SMCC, were eliminated. The purified activated ligand can be prepared in large quantities, stored in freezer at −20° C., and ready for use at the time of conjugation. Thus the necessity for repeated synthesis of activated ligand is avoided.

3) Dimerization of the thiol oligomers was minimized to undetectable level by conducting the thiol oligomer purification and conjugation reaction under strictly degassed condition. Degassed conditions of the present invention shall mean mildly anaerobic conditions, more preferably means low oxygen, and most preferably means no oxygen is present. It is preferable to remove any trace of unreacted reagent and other low molecular weight thiol-containing impurities. This was accomplished by degassing the solvent used in the G-25 purification of the thiol oligomers, by using the freshly collected thiol oligomer. G-25 fraction immediately in conjugation reaction, and by conducting the conjugation in vacuum condition. These precautions, combined with the use of pure SMCC-modified ligand and pure thiol oligomers in the conjugation reaction, formed the foundation for achieving quantitative conjugation of the oligomers.

4) The elimination of side reactions and the resulting quantitative conjugation reaction made it possible to employ a simple G-25 purification method to obtain the pure conjugates. Thus, PAGE purification, as found necessary in Method 1, was eliminated. This gave rise to pure conjugates in greater than 95% overall yields.

5) The optional incorporation of the 3′-tracer unit into the oligomer construct gives rise to extreme flexibility in radiolabeling of the final conjugates of all backbone types. A conjugate can be prepared and stored in large quantity at one time and can be labeled later whenever it is needed, e.g., before its use in cellular uptake and biodistribution experiments. This eliminates the necessity for repeated synthesis of the same conjugate in order for its radiolabeling, as was the case in Method 1.

It was due to these advantages of the conjugation Method 2 that it has become our routine conjugation method since its invention. The A-L-P conjugates used in our bioefficacy studies were all synthesized by this method.

### Cellular Uptake Experiments

### Example 4

This example illustrates the materials and methods utilized for cellular uptake experiments Hep G2 cells, Hep G2 2.2.15 cells, HT 1080 cells or HL-60 cells.

Materials: Minimal essential medium with Earle's salts supplemented with L-glutamine (MEM), Dulbecco's modified Eagle's medium (D-MEM), RPMI medium 1640 supplemented with L-glutamine (RPMI), Dulbecco's phosphate buffered saline (D-PBS), fetal calf serum (FCS), sodium pyruvate (100 mM), non-essential amino acids (10 mM), aqueous sodium bicarbonate (7.5%), and trypsin (0.25%; prepared in HBSS with 1.0 mM EDTA) were purchased from GIBCO BRL. Human hepatocellular carcinoma (Hep G2) (ATCC HB 8065), human fibrosarcoma (HT 1080), and human promyleocytic leukemia (HL-60) cells were purchased from ATCC. Hep G2 2.2.15, a human hepatocellular carcinoma cell line stably transfected with human hepatitis B virus DNA (HepG2 2.2.15) (Sells, et al., (1987), Proc. Natl. Acad. Sci., 84:1005-1009), was a gift of Dr. G. Y. Wu. Other lines of suitable cells are known to persons of skill in the art, for example PLC/PRF/5 (Alexander cells), a human hepatoma secreting hepatitis B surface antigen, has been described (Jacinta, S., (1979), Nature, 282:617-618) and is available from the American Type Culture Collection.

The cells were maintained in 1×MEM supplemented with 10% fetal calf serum (FCS), 1 mM sodium pyruvate, and 0.1 mM non-essential amino acids or 1× RPMI supplemented with 10% FCS (Hep G2), 1× RPMI supplemented with 10% FCS (HepG2 2.2.15), 1× D-MEM supplemented with 10% FCS (HT-1080), or 1× RPMI supplemented with 10% FCS (HL-60). Silicon oil was initially a gift from General Electric (#SF 1250) and subsequently purchased from Nye Lubricants Inc (# 98-0704). Cells were counted using a Coulter Cell Counter purchased from Coulter Electronics

Methods: Hep G2, Hep G2 2.2.15, and HT 1080 cells were passaged into 2 cm is wells and grown in the appropriate medium to a density of about 2-4×10 cells per well. The maintenance media was aspirated and the cells were incubated at 37° C. with 0.5 mL medium that contained 2% FCS and was made 1 μM in [5′-³²P]-labeled 10. After the prescribed time had elapsed, a 5 uL aliquot of the media was saved for scintillation counting and the remainder aspirated from the well. The cells were washed with D-PBS (2×0.5 mL), treated with 0.25% trypsin (37° C., 2 minutes) and suspended in fresh growth medium containing 10% FCS. The suspended cells were layered over silicon oil (0.5 mL) in a 1.7 mL conical microcentrifuge tube and pelleted by centrifugation at 14,000 rpm (12,000 g) for 30 seconds. The supernatant was carefully decanted and the cell pellet was lysed with 100 uL of a solution containing 0.5% NP 40, 100 mM sodium chloride, 14 mM Tris (HCl and 30% acetonitrile). The amount of radioactivity, and by inference the amount of 10 associated with the cell lysate, was determined by scintillation counting.

RPMI medium supplemented with 2% FCS and made 1 μM in [³²P]-10 was pre-treated with 7.5×10⁶ HL 60 cells for 5 minutes at room temperature. The cells were removed by centrifugation (5 minutes). The medium was decanted and added to 7.5×10⁶ fresh HL 60 cells. The cells were evenly suspended and cell suspension divided into six 0.4 mL-portions. The remainder was discarded. The cells were incubated for the prescribed time, then collected by centrifugation (5 minutes), resuspended in 0.5 mL D-PBS and layered onto silicon oil in a 1.7 mL conical microfuge tube. The cells were pelleted by centrifugation (12,000 g, 30 seconds), lysed, and the amount of [³²P]-labeled material associated with the cells determined by scintillation counting.

### Example 5

This example illustrates the uptake of 10 by HepG2 cells in vitro. In this case 10 was synthesized utilizing Conjugation Method 1.

The cellular association of the conjugate 10 was examined, both alone and in the presence of 100 equivalents of free neoglycopeptide 5, with Hep G2 cells to demonstrate that uptake by the cells was a result of binding of the neoglycopeptide moiety of 10 to the hepatic carbohydrate receptor. As a control, an oligo-mp modified at the 5′-end with ethylenediamine (FIG. 2) was also incubated with Hep G2 cells under identical conditions. Modification of the 5′-phosphate with ethylenediamine was accomplished by incubation of 5′-phoshorylated 2 with 0.1 M EDAC in a buffer containing 0.1 M imidazole at pH 7 at 37° C. for 2 hours followed by overnight incubation with an aqueous solution 0.3 M ethylenediamine hydrochloride buffered to pH 7.0. (Miller, P. S.; Levis, J. T., unpublished results). This modification prevents removal of the 5′-phosphate by cellular phosphatase activity.

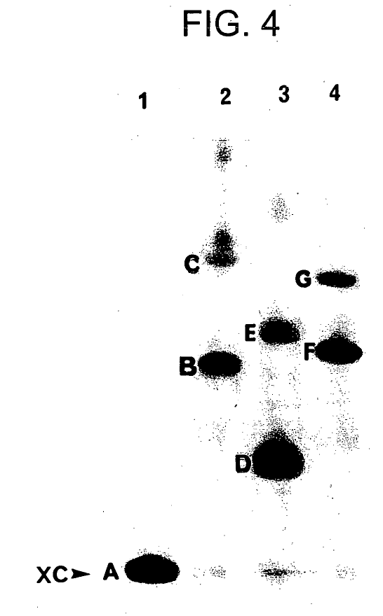

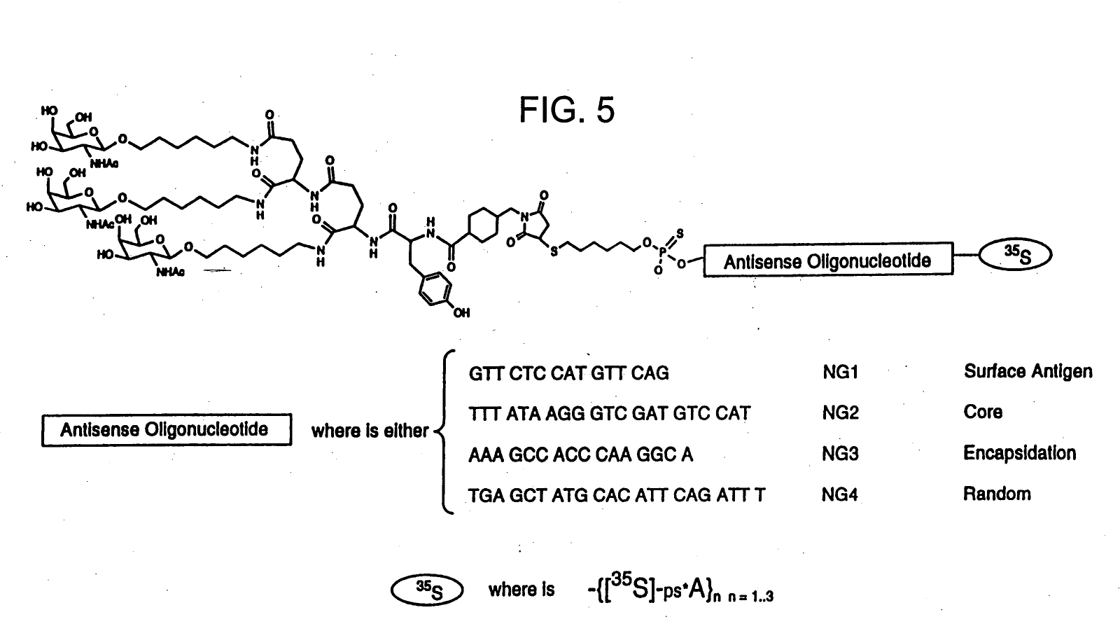

In each instance, the modified oligo-mp was present at a concentration of 1 μM in medium containing 2% fetal calf serum (FCS) and incubations were carried out at 37° C. The conjugate rapidly associated with the cells when incubated alone, loading the cells in a linear fashion to the extent of 7.8 pmol per 10⁶ cells after only two hours (FIG. 6). In contrast, when a 100-fold excess of free 5 was present with 1 μM conjugate, association of 10 was only 0.42 pmol per 10⁶ cells, a value essentially identical to that obtained with the control oligo-mp 6b, which does not contain the neoglycopeptide (0.49 pmol per 10⁶ cells). As an additional control; Hep G2 cells were incubated with 6b in the presence of a 10-fold excess of 5 to assess the possibility that despite the absence of a covalent link between 5 and 6b, 5 could cause uptake of 6b by the Hep G2 cells. The amount of cell associated-6b following a two-hour incubation was only 0.60 pmol per 10⁶ cells, significantly less than found with the conjugate 10. In addition, the uptake of 10 by Hep G2 cells for longer times was examined (1 μM conjugate, 37° C.), and found to be linear up to about 24 hours reaching a value of 26.6 pmol per 10⁶ cells (FIG. 7). The results of these experiments indicate that: (1) the conjugate 10 associates with Hep G2 cells by binding specifically to the asialoglycoprotein receptor; (2) a covalent link between the oligo-mp and neoglycopeptide is essential for significant enhancement of the association of the oligo-mp with Hep G2 cells; and (3) uptake of 10 by Hep G2 cells does not appear to saturate up to 24 hours under the conditions used in this study.

### Example 6

This example illustrates the specificity of 10 for cells of hepatic origin (Hep G2).

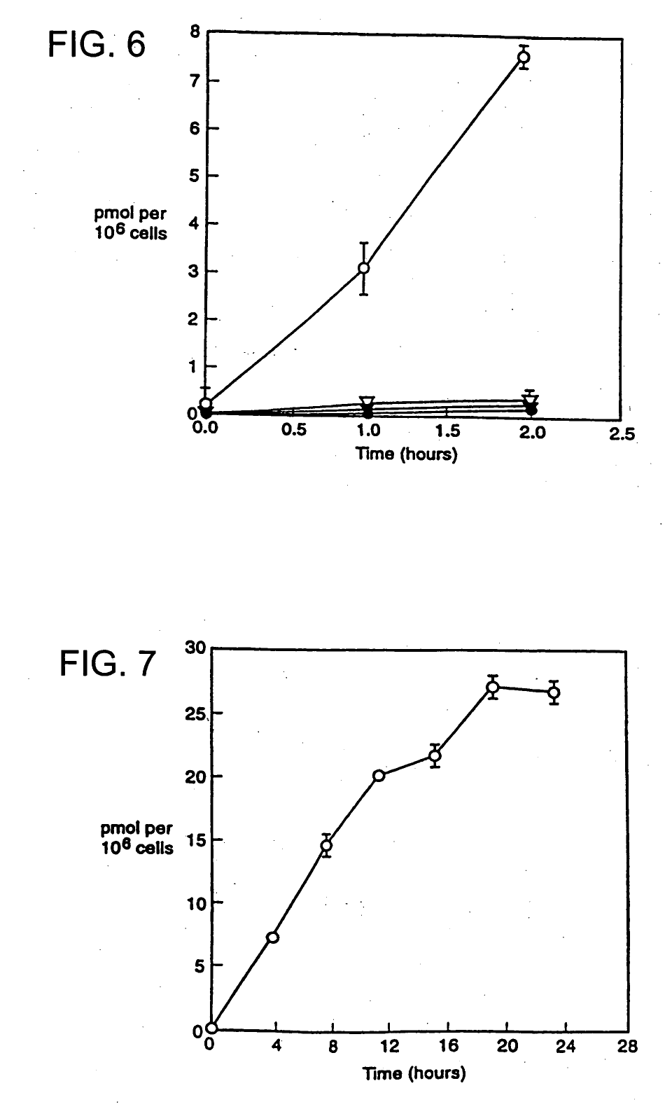

Cell-type specificity of the compounds was also examined. It is well established that the asialoglycoprotein receptor is found on the surface of hepatocytes and represents an efficient means for selectively targeting this tissue for delivery of a variety of therapeutic agents (Wu and Wu, (eds.), (1991), in Liver Diseases, Target Diagnosis and Therapy Using Specific Receptors and Ligands, Marcel Dekker, Inc., New York). Tissue specificity was examined by incubating three human cell lines, Hep G2, HL-60 and HT 1080, in medium containing 1 uM conjugate 10 and 2% FCS at 37° C. for 3 and 24 hours. The only cell line to exhibit significant uptake of 10 was Hep G2. After incubation for 3 and 24 hours, 8.5 and 26.7 pmol per 10⁶ cells, respectively, was associated with the Hep G2 cells (FIG. 8). In contrast, after 24 hours only 0.10 and 0.53 pmol per 10⁶ cells were associated with the HL-60 cells and HT 1080 cells, respectively.

### Example 7

### FIG. 9

This example illustrates the uptake of the liver specific neoglycoconjugate containing oligomers comprised of other nuclease resistant backbones.

The above examples illustrate that OMNP's can be conjugated to the hepatic specific ligand YEE(ah GalNAc)₃ to yield a homogeneous and defined neoglycoconjugate. Furthermore, this neoglycoconjugate is taken up by hepatoma-derived cells (Hep G2) specifically and at an enhanced rate in vitro. The above results have been extended to consider oligonucleotides with other nuclease resistant backbone modifications, such as phosphorothioates (ps) oligomers comprised of 2′Omethyl ribose moieties and alternating phospho-diester/methylphosphonate linkages (2′Ome-po/mp). The experimental methods were identical to those utilized in Examples 4 and 5. Results of these experiments were very similar to those observed with the OMNP containing neoglyco-conjugates. Neoglycoconjugate containing phosphorothiate oligomers were synthesized according to Conjugate Method 2. YEE (ahGalNAc)₃-SMCC-ps ^{5′}GTTCTCCATGTTCAG^{3′} (NG-1) was labeled with ³⁵S using the 3′-end labeling method described in Conjugation Method 2 displayed a linear uptake to the extent of 17.25 pmoles/10⁶ cells at 24 hours. In contrast the corresponding unconjugated oligomer ps ^{5′}GTTCTCCATGTTCAG^{3′} was taken up by Hep G2 cells at a diminished rate, reaching 1.01 pmoles/10⁶ cells at 24 hours. In a similar fashion, neoglyco-conjugates containing 2′ OMe alternating po/mp oligomers (YEE(ahGalNAc)₃-SMCC-2′OMe ^{5′}AGₚUCₚAGₚUCₚAGₚUCₚAGₚU^{3′}) displayed a linear uptake to the extent of 24.3 pmoles/10⁶ cells at 24 hours. The corresponding unconjugated oligomer (2′OMe ^{5′}AGₚUCₚAGₚUCₚAGₚUCₚAGₚU^{3′}) displayed minimal uptake of less than 1 pmole/10⁶ cells at all time points assayed. All oligomers and neoglycoconjugates were stable in cell culture media up to 24 hours. These results illustrate the delivery utility of the unique ligand-linker complex and give us a platform to expand this system to the delivery of other therapeutic agents.

### Example 8

### FIG. 10

This example illustrates the uptake of liver specific neoglycoconjugates containing oligomers comprised of nuclease resistant backbones by Hep G2 2.2.15 cells in vitro.

Hep G2 2.2.15 cells are hepatoma cells that have been stably transfected with the Hepatitis B virus. Cellular uptake of the 2′OMe po/mp and ps oligomers cited in Example 7 both synthesized and labeled by the Conjugation Method 2, were assayed utilizing the methods described in Examples 4 and 5. The results were very similar to the cellular uptake experiments described in Example 7. Neoglycoconjugates containing ps oligomers displayed linear and rapid uptake to the extent of 20 pmoles/10⁶ cells at 24 hours, while the corresponding unconjugated ps-oligomer associated poorly at less than 1.0 pmole/10⁶ cells at 24 hours (FIG. 10). In a similar fashion, neoglyco-conjugates containing 2′OMe po/mp oligomers were taken up by Hep G2 2.2.15 cells in a rapid and linear rate to the extent of 28.52 pmoles/10⁶, while less than 1 pmole/10⁶ cells of the corresponding unconjugated oligomer was taken up after 24 hours incubation. Stability of the neoglycoconjugates and the unconjugated oligomers in cell culture media was determined by poly-acrylamide gel electrophoresis. Degradation products were not detected in either case for up to 96 hours incubation at 37° C.

The cellular uptake experiments previously described utilizing ³²P-labeled oligo-mp conjugates were extended to examine the cellular association of neoglycoconjugates comprised of neoglycopeptide 5 and oligomers of other nuclease resistant backbones, most notably ps and 2′OMe po/mp, with Hep G2 cells. Neoglycoconjugates containing a phosphorothioate oligomer, YEE(ahGalNAc)₃-SMCC-ps ^{5′}GTTCTCCATGTTCAG^{3′} (NG-1) was labeled using Conjugation Method 2, which displayed linear uptake to the extent of 17.25 pmoles/10⁶ cells at 24 hours. In contrast, the corresponding unconjugated oligomer ps ^{5′}GTTCTCCATGTTCA-G^{3′} was taken up by Hep G2 cells at a diminished rate, reaching 1.01 pmoles/10⁶ cells at 24 hours. In a similar fashion, neoglyco-conjugates containing 2′OMe alternating po/mp oligomers (YEE(ahGalNAc)₃-SMCC-2′OMe ^{5′}AGₚUCₚAGₚUCₚAGₚUCₚAGₚU^{3′}) displayed a linear uptake to the extent Of 28.52 pmoles/10⁶ cells at 24 hours (FIG. 9; Table 6). The corresponding unconjugated oligomer (2′OMe ^{5′}AGₚUCₚAGₚUCₚAGₚUCₚAGₚU^{3′}) displayed minimal uptake of less than 1 pmole/10⁶ cells. These results illustrate the delivery utility of the unique ligand-linker complex and allow a platform to expand this system to the delivery of other therapeutic agents.

The enhanced cellular uptake observed in Hep G2 cells is also evident in Hep G2 2.2.15 cells (FIG. 10a). Neoglycoconjugates containing ps oligomers displayed linear and rapid uptake to the extent of 20 pmoles/10⁶ cells at 24 hours, while the corresponding unconjugated ps-oligomer associated poorly at less than 1.0 pmole/10⁶ cells at 24 hours. In a similar fashion, neoglyconjugates containing 2′OMe po/mp oligomers were taken up by Hep G2 2.2.15 cells in a rapid and linear rate to the extent of 28.97 pmoles/10⁶ (FIG. 10b; Table 7), while less than 1 pmole/10⁶ cells of the corresponding unconjugated oligomer was taken up after 24 hours incubation. Similar results have been observed by investigators using other liver specific ligands and have led to the conclusion that stable transfection with HBV does not alter receptor activity in these cells (Wands et. al., 1997, supra). These delivery systems, however, had been demonstrated to deliver charged sa-oligomers only. The liver specific ligand used in this report has been shown to have increased utility in the sense that it can enhance cellular uptake of uncharged ONMP's, charged sa-oligomers and half-charged 2′-OMe ONMP/phosphodiester alternating oligomers with a similar degree of effectiveness.

TABLE 6
Uptake of conjugated YEE (ah-GAlNac)₃-SMCC-AET-2′O-Me
^{5′}AGₚUCₚAGₚUCₚAGₚUCₚAGₚU^{3′} (1d) and EDA-2′-0 -Me-
^{5′}AGₚUCₚAGₚUCₚAGₚUCₚAGₚU^{3′} (1e) by Hep 2G 2.2.15 cells in
culture (pmoles/10⁶ cells)

| OLIGOMER | 1 HOUR | 2 HOURS | 3 HOURS | 24 HOURS |
|---|---|---|---|---|
| 1d | 3.63 | 7.71 | 14.16 | 28.52 |
| 1e | 0.277 | 0.305 | 0.400 | 0.450 |

TABLE 7
Uptake of YEE(ah-GAlNac)₃-SMCC-S(CH₂)₄-ps-2′O-Me-
^{5′}AP_{g}UCₚAGₚUCₚAGₚUCₚAPₚU^{3′}-U^{M}dT*^{3′-3′} (dT-T) -³²P-EDA
(1c) by Hep G2 2.2.15 cell in culture (pmoles/10⁶ cells)

| | | | | 16 | 28 |
|---|---|---|---|---|---|
| OLIGOMER | 4 HOURS | 8 HOURS | 12 HOURS | HOURS | HOURS |
| 1c | 9.44 | 18.60 | 22.05 | 24.92 | 28.97 |

### Whole Animal Biodistribution and Pharmacokinetics

### Example 9

This example illustrates the materials and methods utilized in whole animal experiments using a ³²P-labled A-L-P conjugate (10) as an example. Materials: Dulbeccos phosphate buffered saline pH 7.2 was purchased from Meditech, (Sterling, Va.). Solvable tissue solubilizer was purchased from Life Technologies, (Grand Island, N.Y.). Cytoscint scintillation fluid was purchased from ICN (Costa Mesa, Calif.). Scintillation vials were purchased from Kimble Glass, (Vineland, N.J.). Anhydrous ether was purchased from J.T. Baker, Sanford, M E. CD-1 male mice (22-35 grams in weight) were obtained from Charles River (Wilmington, Mass.). Centricon filters (30,000 MWC) were obtained from (Millipore, Bedford, Mass.). Tissue samples were homogenized with a Polytron homogenizer Model PCU 2-110 (Brinkman Inst., Westbury, N.Y.).

Methods: Briefly, the parent oligodeoxynucleoside methylphosphonate (oligo-MP), U^{m}pT₇, was 5′ end-labeled with [³²P)-ATP and ATP to give p*UmpT7 having a specific activity of 300 μCi/14 nmol (the * indicates the position of the radioactive nuclide). The 5′ phosphate was modified with cystamine in the presence of 1-methylimidazole and water-soluble carbodiimide. The resulting disulfide was reduced with excess dithiothreitol and conjugated with the ligand, YEE(ahGalNAc)₃, using the heterobifunctional cross-linking reagent SMCC. The conjugate 10, [YEE(ahGalNAc)₃]-SMCC-AET-pU^{m}pT₇, was purified by polyacrylamide gel electrophoresis, extracted from the gel and desalted using a SepPak cartridge. The pure conjugate was characterized both enzymatically and chemically. A portion of the conjugate was treated with N-acetylglucosamidase in order to completely remove the GalNAc residues to give 11, [YEE(ah)₃]SMCC-AET-pU^{m}pT₇, (Trubestskoy, et al., (1992), Bioconjugate Chem., 3:323-327). Both 10 and 11 were >99% pure as judged by PAGE analysis. The solutions containing the conjugates were placed in sterile test tubes and lyophilized under aseptic conditions in preparation for the whole animal biodistribution and pharmacokinetic experiments.

The conjugates 10 and 11 were redissolved in sterile water. Male CD-1 mice (Charles River), weighing 22 to 35 g, received a single injection via tail vein of 7-30 picomoles of [³²P]-[YEE(ahGalNAc)₃]-SMCC-AET-pU^{m}pT₇ (10) or 7 pmole of ³²P]-[YEE(ah)₃] SMCC-AET-pU^{m}pT₇ (11) contained in 0.2 mL of saline. The mice were sacrificed by cervical dislocation at 15, 30, and 60 minutes and 2, 4, 6, and 24 hours. Blood, liver, kidneys, spleen, muscle, upper and lower gastrointestinal tract and feces were collected and weighed. Representative samples from these organs and tissues were weighed and placed in glass vials. In order to collect the urine (2 hours post injection), the external urethra of the mice was ligated under short ether anesthesia and, after sacrifice, the bladders were removed and placed into glass vials. Solvables® (NEN; 1 mL) was added to each sample. The samples were then placed on a slide warmer to be digested overnight and removed the next morning to cool. The digested samples were decolorized with 3 to 7 drops of H₂O₂ (30% w/v), and 10 mL Formula 989 (NEN) scintillation cocktail were added. The amount of radioactivity was determined by scintillation counting (Packard 1900 TR; <3% error). Aliquots of the injected dose were counted along with the samples to calculate the percent dose per organ or gram tissue.

Male CD-1 mice, weighing between 22 to 35 g, received a single injection via tail vein of 40 pmole of [³²P]-[YEE(ahGalNAc)₃]SMCC-AET-pU^{m}pT₇ (10). Animals were sacrificed after 15, 60 and 120 minutes. Livers and bladders were collected as before, placed into plastic vials and immediately frozen at −80° C. Samples of liver were thawed to 0° C. and weighed (average mass 0.25 g). The tissue was homogenized (Polytron PCU-2-110 Tissue Homogenizer) in 4 volumes of acetonitrile/water (1:1). Tissue debris was removed by centrifugation (10,000 g, 20 minutes, 0° C.; Sorval Model RC-5B Refrigerated Superspeed Centrifuge). The supernatent was removed and the extraction procedure repeated. Typical recovery of radioactivity from the liver samples was 90% as judged by comparison of aliquots of decolorized homogenate and supernatant. A portion of the supernatent was filtered through a Centricon filter (30,000 MWC; 20 minutes, 0° C., 10,000 g; Herml Z 360 K Refrigerated Microcentrifuge) and lyophilized. The residue was redissolved in 10 mL formamide loading buffer (90% formamide, 10% 1×TBE, 0.2% bromophenol blue, and 0.2% xylene blue) in preparation for analysis by polyacrylamide gel electrophoresis (PAGE; 15%, 20×20×0.75 cm, 2 V/cm, 45 minutes). The urine was collected from the bladder, which had been thawed to 0° C., and was deproteinized with ethanol (1:2 v/v) at 0° C., for 30 minutes. The precipitated proteins were removed by centrifugation (16,000 g, 20 minutes, 0° C.,). Recovery of radioactivity was estimated to be 90% by comparing the aliquots of the supernatent and the protein pellet. A portion of the supernatant was lyophilized, redissolved in formamide loading buffer and analyzed by PAGE (15%, 20×20×0.75 cm, 2 V/cm, 45 min). Standards were produced by incubation of full-length conjugate 10 with, in separate reactions, N-acetylglucosamidase in 50 mM sodium citrate, pH 5.0, chymotrypsin in 10 mM Tris(HCl containing 200 mM KCl, pH 8.0 and 0.1 N HCl each at 37° C. for 30 minutes.

Cells (about 10⁵) were incubated in media containing 1 μM [³²P]-labeled 10 for 2, 4, 8, 16, and 24 hours, washed with PBS (2×), pelleted through silicon oil and lysed (0.5% NP 40, 100 mM sodium chloride, 14 mM Tris-HCl pH 7.5, 30% ACN). The lysate was extracted with 50% aqueous acetonitrile (v/v) twice. The extracts were lyophilized, redissolved in formamide loading buffer and analyzed by PAGE (15%, 2 V/cm, 30 minutes).

### Example 10

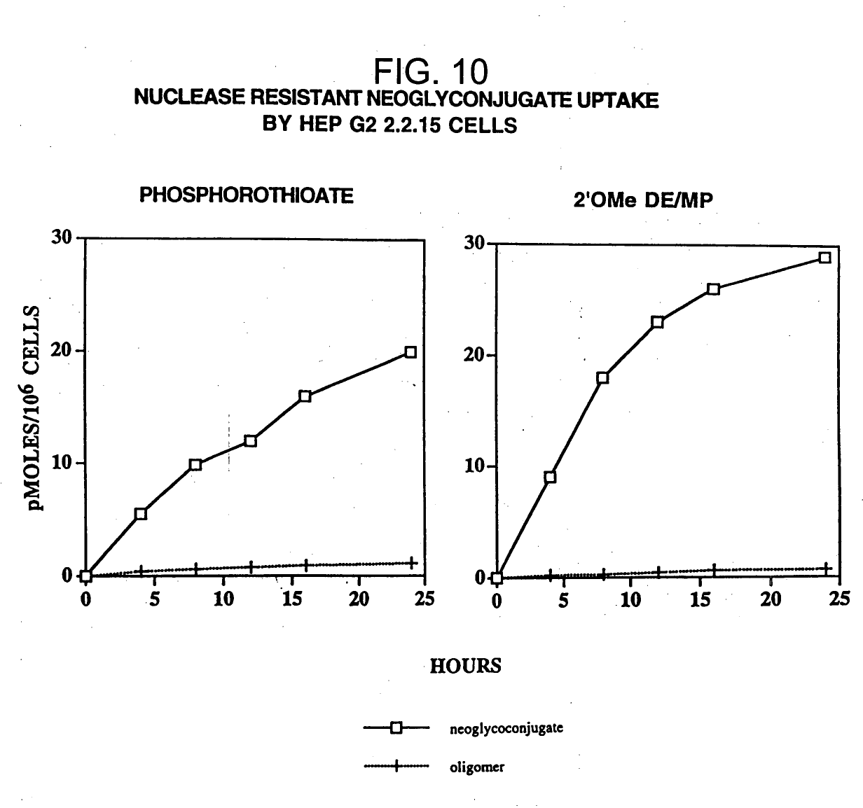

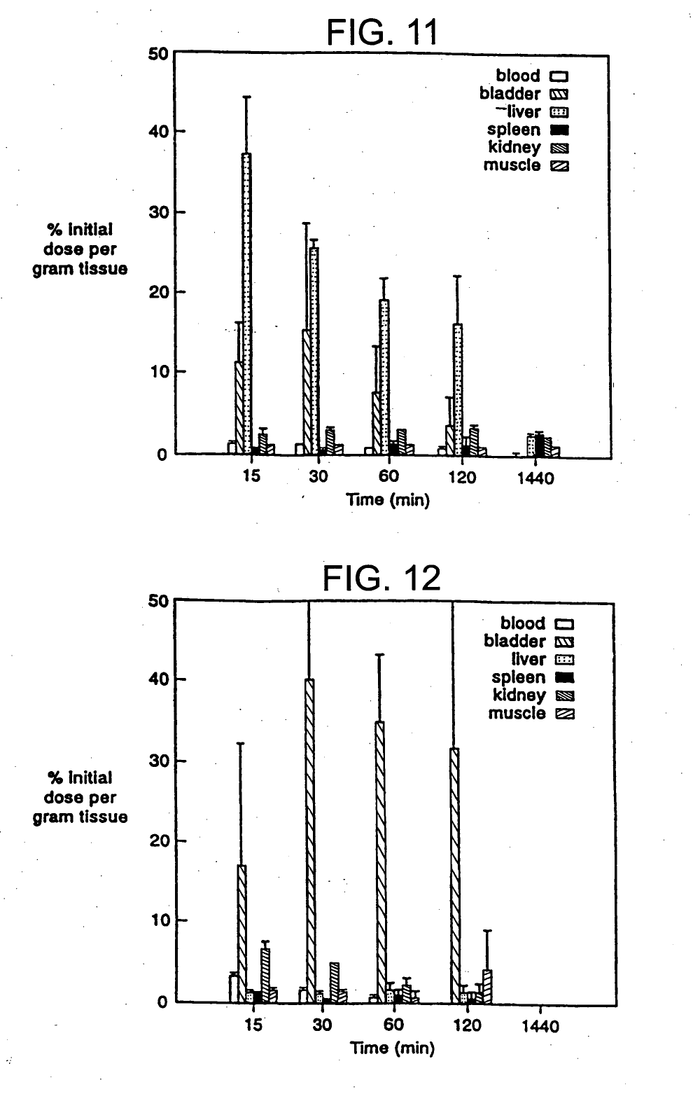

This example illustrates whole animal experiments that were performed to test for the ability of a delivery vehicle containing the asialoglycoprotein ligand, YEE(ahGalNAc)₃, and radiolabeled with ³²P, to deliver synthetic oligo-MPs specifically to the liver of mice (FIGS. 11 and 12).

For comparison, a conjugate lacking the three terminal GalNAc residues was also synthesized and tested. This sugarless conjugate served as a control for the study of ligand (GalNAc)-specific uptake in mice.

In order to investigate the in vivo tissue and organ distribution of conjugate 10, mice were injected via tail vein with radiolabeled conjugate as described above and the amount of radioactivity associated with each organ determined by scintillation counting. Table 8 shows the conjugate associates to the greatest extent with the liver, reaching a value of 69.9% of the injected dose 15 minutes post-injection. The ranking of total radioactivity in the other tissues measured at 15 minutes post-injection was, in decreasing order: muscle>kidney>blood>spleen. The peak value of radioactivity for the urine was 28% of the injected dose and was reached after 30 minutes. The amount of radioactivity associated with the kidneys and blood decreased over time. It is noteworthy that, while it may be expected that metabolites of the conjugate produced in the liver would become deposited in the gastrointestinal tract via bile excretion, little radioactivity was associated with the gall bladder, upper and lower gastrointestinal tract, and feces. Similar results were observed when mice were injected with a low dose (7 pmoles) of neoglycoconjugate 10 (FIG. 11; Table 9).

Table 10 shows that conjugate 11, which lacks the three terminal GalNAc residues, was distributed in the order: muscle>blood>kidneys>liver>spleen. The amount of muscle and liver radioactivity appeared to remain constant whereas that associated with the blood and kidneys decreased over the 24 hour study. The peak value of radioactivity in the urine was 39.9% at 30 minutes post-injection (FIG. 12).

### Example 11

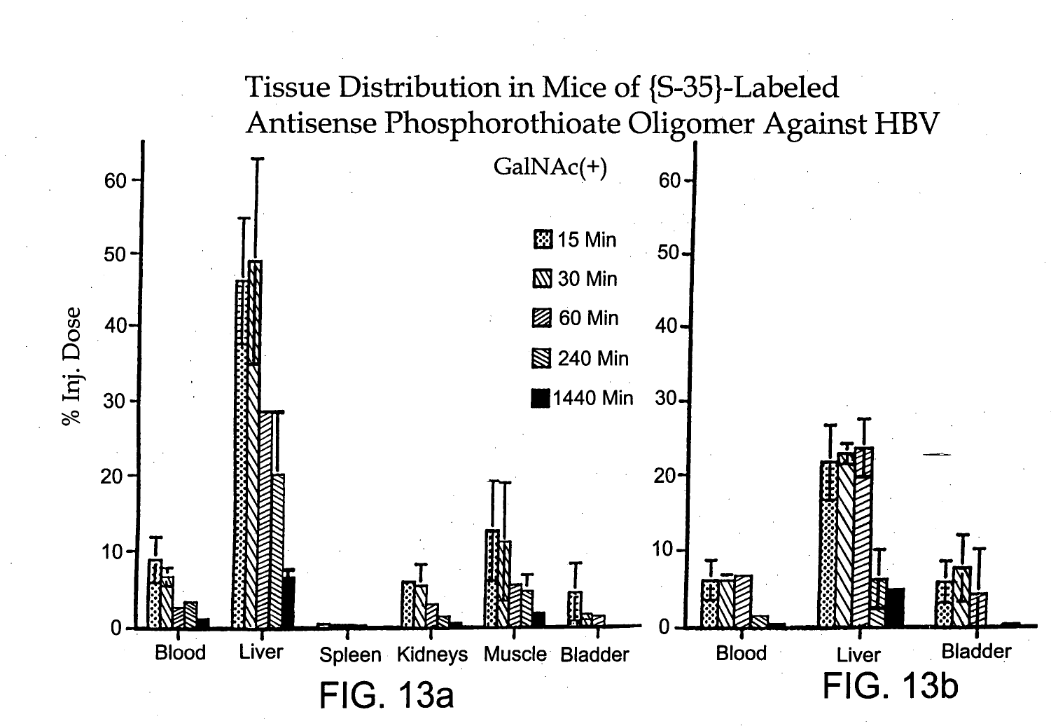

This example illustrates whole animal experiments that were performed to test for the ability of a delivery vehicle of the invention, i.e., which contains the asialoglycoprotein ligand, YEE(ahGalNAc)₃, and radiolabeled with ³⁵S, to deliver synthetic, nuclease resistant phosphorothioate oligomers specifically to the liver of mice (FIG. 13).

Male CD-1 mice were injected as described in Example 9 with 30 pmoles of the neoglycoconjugate YEE(ahGalNAc)₃-SMCC-ps-(TTTATAAGGGTCGATGTCCAT)-^{{35S}}(psA)ₙ labeled utilizing the 3′-end labeling method as decribed in Conjugation Method 2. For comparison, a conjugate which lacks the three terminal GalNAc residues, YEE(ah)₃-SMCC-ps-(TTTATAAGGGTCGATGTCCAT)-(psA)ₙ^{{35S}} was also synthesized. This sugarless conjugate served as a control for the study of ligand (GalNAc)-specific uptake in mice. Experimental results were very similar to those observed in Example 10. The conjugate containing the terminal, sugar residues associated to the greatest extent with the liver, reaching a value of 46.19% of the injected dose 15 minutes post-injection. The ranking of total radioactivity in the other tissues measured at 15 minutes post-injection was, in decreasing order: muscle>blood>kidney>spleen. The peak value of radioactivity for the urine was 4.51% of the injected dose and was reached after 15 minutes. The amount of radioactivity associated with the kidneys and blood decreased over time.

The conjugate, which lacks the three terminal GalNAc residues, was taken up at a reduced rate by the liver reaching a peak of 23.67% of the injected dose at 30 minutes. This conjugate was cleared from the blood and urine within 4 hours.

### Example 12

This example illustrates the polyacrylamide gel electrophoresis analysis of the metabolism of conjugate 10 isolated from mouse liver and Hep G2 cells.

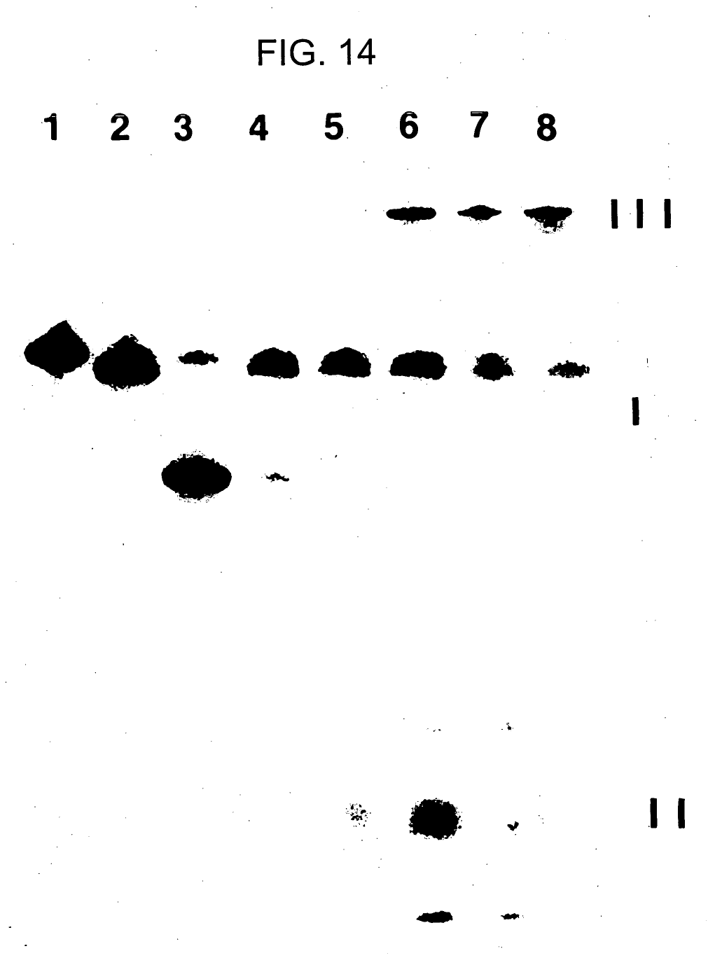

FIG. 14 shows the results of PAGE analysis of the metabolism of conjugate 10 following incubation with Hep G2 cells for 2 to 24 hours. Three classes of metabolites are identified (FIG. 14; labeled I-III) according to their electrophoretic mobility versus control reactions. Class I appears to consist of

TABLE 8
Kinetics of (³³P)-(Yee(ah-GalNac)₃)-SMCC-AET-pU*pT₇,
in mice infected i.v. at a dose level of 30 p mol.

| | percent injected does per organ | | | | | | |
|---|---|---|---|---|---|---|---|
| | injection (min.) | | | | | | |
| Organ | 15 | 30 | 60 | 120 | 240 | 360 | 1440 |
| Blood^{a} | 2.79 ± 0.18 | 2.25 ± 0.45 | 1.42 ± 0.38 | 0.90 ± 0.26 | 1.09 ± 0.16 | 1.23 ± 0.30 | 0.61 ± 0.11 |
| Liver^{a} | 69.9 ± 9.9 | 41.8 ± 9.3 | 25.2 ± 2.4 | 14.2 ± 2.2 | 10.6 ± 4.2 | 8.5 ± 0.6 | 3.2 ± 1.4 |
| Spleen^{a} | 0.08 ± 0.04 | 0.05 ± 0.03 | 0.2 ± 0.01 | 0.17 ± 0.04 | 0.24 ± 0.02 | 0.16 ± 0.08 | 0.25 ± 0.04 |
| Kidney^{a} | 3.00 ± 1.26 | 2.12 ± 0.27 | 1.58 ± 0.15 | 1.26 ± 0.19 | 1.25 ± 0.21 | 1.80 ± 0.70 | 0.92 ± 0.19 |
| Muscle^{a} | 7.53 ± 1.49 | 8.42 ± 1.51 | 8.46 ± 2.32 | 8.76 ± 0.92 | 13.0 ± 3.9 | 17.2 ± 4.6 | 13.9 ± 1.3 |
| Upper | 3.63 ± 1.85 | 12.72 ± 9.41 | 6.28 ± 1.74 | 3.73 ± 2.80 | 3.19 ± 0.78 | 3.92 ± 0.97 | 2.01 ± 0.28 |
| G.I. | | | | | | | |
| Lower | 0.24 ± 0.05 | 0.33 ± 0.20 | 0.38 ± 0.14 | 0.34 ± 0.05 | 0.63 ± 0.26 | 0.50 ± 0.22 | 0.48 ± 0.09 |
| G.I. | | | | | | | |
| Gall | 0.27 ± 0.23 | 0.62 ± 0.14 | 0.7^{b} | 0.4^{c} | 0.31^{c} | 0.17^{c} | NA |
| Bladder | | | | | | | |
| Feces^{b} | 0.01 ± 0.01 | 0.05 ± 0.05 | 0.05 ± 0.03 | 0.27 ± 0.26 | 1.40 ± 1.11 | 0.47 ± 0.23 | 0.55 ± 0.41 |
| ^{a}Values are reported as the average percent injected dose per organ in three animals ± one standard deviation. Approximately 0.5 microCi (30 pmol) intravenously into each mouse. The mass of each organ was determined separately and was used to determine the percent dose per organ from percent of conjugate was injected dose per gram of tissue. Typical values for the mass of each organ or tissue are: | | | | | | | |
| #blood = 0.07 × body mass; liver = 1.6 + 0.21 g; spleen = 0.17 + 0.05 g; kidneys = 0.6 ± 0.1 g; muscle = 0.4 × body mass. The average body mass was 32.4 ± 2.0 g (std., dev.; n = 21). The peak value of radioactivity in the urine was 27.7 ± 20.2% of injected done at 60 minutes. The large standard deviation reflects the variation in urine | | | | | | | |
| #production and completeness of collection between individual animals. | | | | | | | |

^{b}Value is from a single determination.
^{c}Value is the average of two independent determinations.

TABLE 9
Percent injected dose accumulated per organ following intravenous injection
of [³²p]-[YEE(ah-GalNac)₃)]-SMCC-AET-pU^{m}pT₇.

| | ¹percent injected does per organ | | | | |
|---|---|---|---|---|---|
| | time post injection (min.) | | | | |
| Organ | 15 | 30 | 60 | 120 | 1440 |
| Blood^{a} | 1.71 ± 0.32 | 1.55 ± 0.23 | 0.87 ± 0.12 | 1.00 ± 0.37 | 0.44 ± 0.13 |
| Liver^{a} | 42.4 ± 8.0 | 28.9 ± 0.97 | 21.7 ± 3.0 | 18.6 ± 6.5 | 2.89 ± 0.45 |
| Spleen^{a} | 0.04 ± 0.02 | 0.08 ± 0.01 | 0.16 ± 0.03 | 0.23 ± 0.04 | 0.30 ± 0.11 |
| Kidneys^{a} | 0.93 ± 0.35 | 1.17 ± 0.11 | 1.18 ± 0.06 | 1.15 ± 0.13 | 0.68 ± 0.13 |
| Muscle^{a} | 9.95 ± 1.04 | 8.37 ± 1.26 | 8.85 ± 1.30 | 8.62 ± 0.97 | 8.63 ± 1.16 |
| ^{a}Values are reported as the average percent injected dose per organ three animals ± one standard deviation. Approximately 0.1 microCi (7 pmol) intravenously into each mouse. The following values were used to determine the percent dose per organ from percent dose per gram of tissue; mass of blood = 0.07 × body mass; mass of liver = 1.14 g; mass of spleen = 0.124 g; | | | | | |
| #mass of kidneys = 0.4 g; mass of muscle = 0.4 × body mass. The average body mass was 23.7 + 1.2 (std. dev.; n = 15). The peak value of radioactivity in the urine was 17.1 ± 10.2% of injevted dose at 30 minutes. The large standard deviation reflects the variation in urine production and completeness of collection between individual animals. | | | | | |

TABLE 10
Kinetics of [³²P]-[YEE(ah)₃]-SMCC-AET-pU⁰³pT₇, following i.v. injection.

| | percent injected does per organ | | | | |
|---|---|---|---|---|---|
| | time post injection (min.) | | | | |
| Organ | 15 | 30 | 60 | 120 | 1440 |
| Blood^{a} | 4.82 ± 0.27 | 2.35 ± 0.33 | 0.91 ± 0.43 | ND | ND |
| Liver^{a} | 1.06 ± 0.21 | 1.14 ± 0.32 | 1.65 ± 0.91 | 1.38 ± 0.83 | ND |
| Spleen^{a} | 0.07 ± 0.01 | 0.07 ± 0.01 | 0.12 ± 0.07 | 0.08 ± 0.08 | ND |
| Kidneys^{a} | 2.46 ± 0.42 | 1.82 ± 0.03 | 0.88 ± 0.27 | 0.73 ± 0.30 | ND |
| Muscle^{a} | 12.9 ± 2.1 | 13.8 ± 2.4 | 25.8 ± 18.6 | 25.3^{c} | ND |
| ^{a}Values are reported as the average of three animals ± one standard deviation. Approximately 0.1 microCi (7 pmol) of conjugate was injected intravenously into each mouse. The following values were used to determine the percent dose per organ from percent dose per gram of tissue; mass of blood = 0.07 × body mass; mass of liver = 1.14 g; mass of spleen = 0.124 g; | | | | | |
| #mass of kidneys = 0.4 g; mass of muscle = 0.4 × body mass. The average body mass was 23.7 + 1.2 (std. dev.; n = 15). The peak value of radioactivity in the urine was 36.9 ± 13.5% of injected dose at 30 minutes. The large standard deviation reflects the variation in urine production and completeness of collection between individual animals. | | | | | |

^{b}Value is the average of two independent determinations.

four chemically distinct species in which I and II predominate at all time points. Distribution of I and II is approximately 1:1 at the earliest time points shifting to predominantly II at longer incubation times. A third metabolite of this class, which co-migrates with a material produced by chymotrypsin digestion of I, is also observed at each time point. The relative amount of this species remains essentially constant up to the final time point (24 hours) where little remains. A fourth, unidentified species, which has slightly slower mobility than 3, is observed at all time points except for the last. All Class I metabolites appear to gradually decrease in amount by the final time point. Class II metabolites consist of radiolabeled species that have much greater electrophoretic mobility when compared to the Class I species. At least five bands are observed, however, not all of them are present at each time point. For example, bands at the positions of highest and lowest mobilities appear to increase up to the 16 hour time point than decrease at 24 hour. The same behavior is observed for the predominant species. Maximal intensity of this band occurs at 8 hours followed by a gradual decrease to 24 hours. As was observed with Class I metabolites, all Class II metabolites appear to decrease in amount by the 24-hour time point. Class III metabolite(s) are largely immobile in the gel matrix and are, for the most part, retained in the well of the polyacrylamide gel. The intensity of this band increases over time, reaching a maximal value at 24 hours.

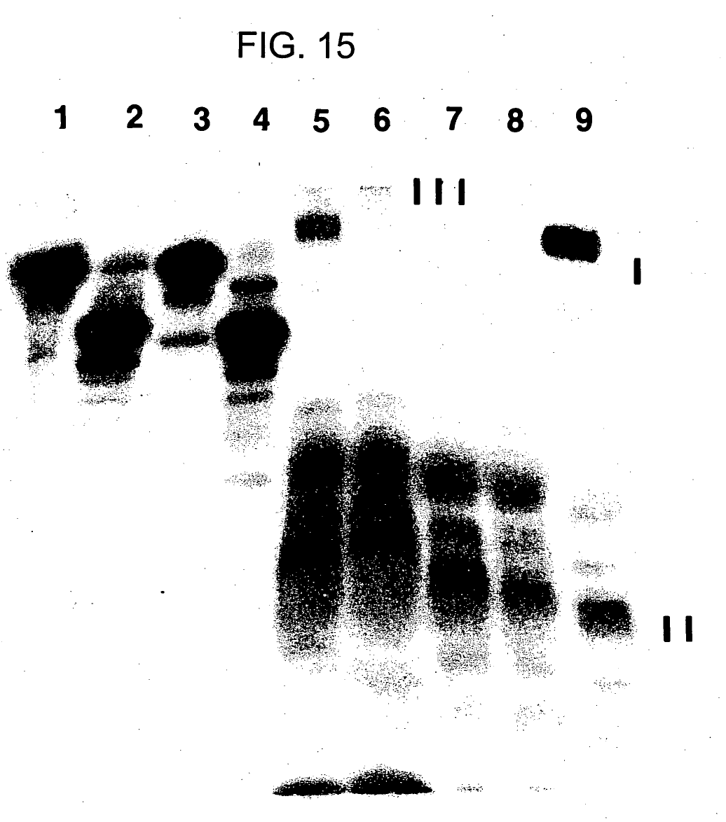

Analysis of the metabolic fate of 10 in intact mouse liver was carried out in a similar fashion. FIG. 15 shows the outcome of PAGE analysis of liver homogenate extracts obtained from liver samples of mice injected with [³²P]-labeled conjugate 10.

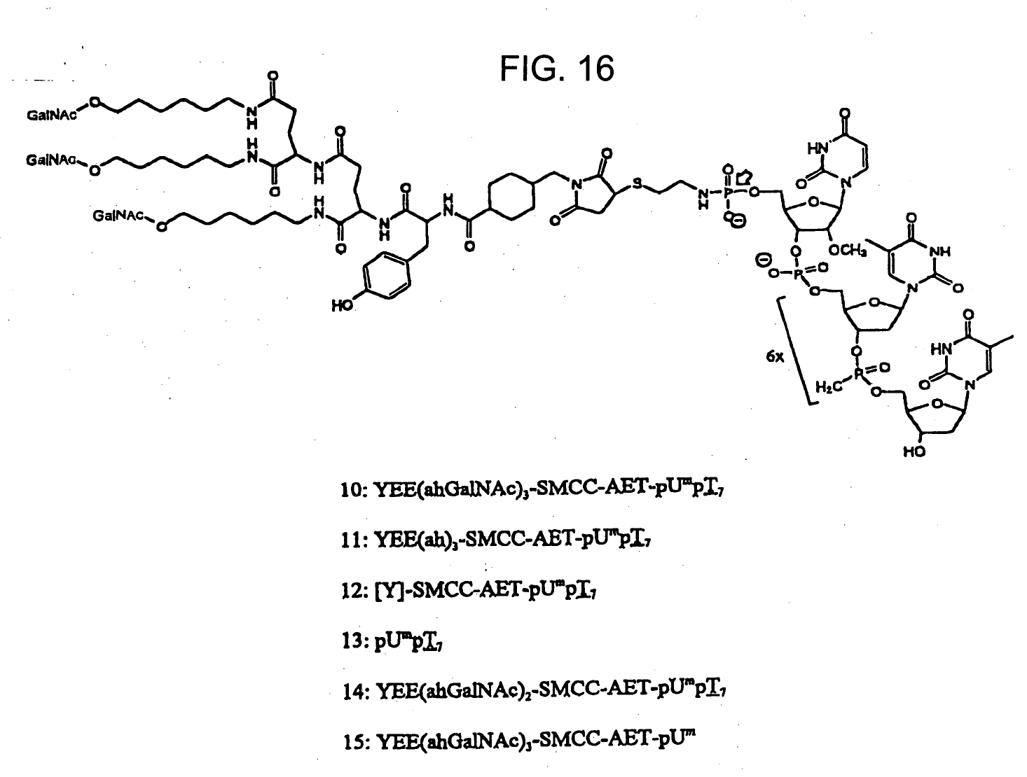

Following 15 minutes post-injection, there remains a significant amount of intact conjugate 10 (FIG. 14; Class I metabolites). The resolution of the gel is not sufficient to permit discrimination between the two species. The remainder of the radiolabeled species in this sample migrated significantly faster than I and II and did not co-migrate with any of the controls. These metabolites appear to have a broader range of mobilities and the slowest are significantly less mobile than the Class II metabolites identified with Hep G2 cells (Class II′). At the later time points, nearly all intact 10 (FIG. 16) has disappeared, whereas the Class II′ metabolites appear to increase in amount.

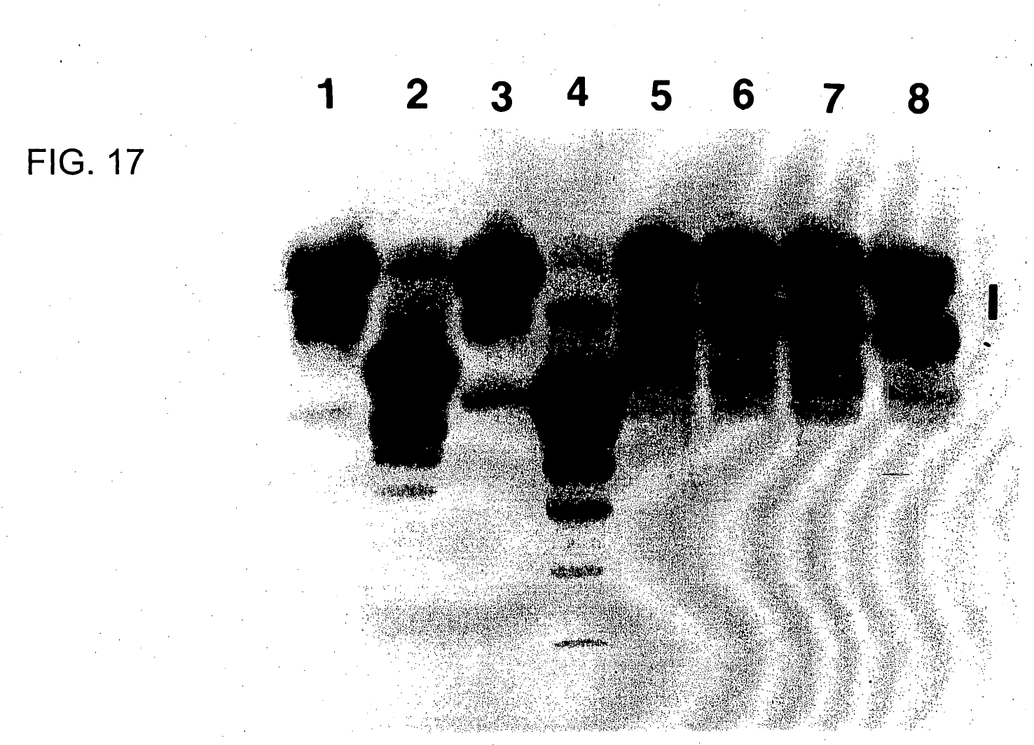

FIG. 17 shows the pattern of metabolites observed in mouse urine following i.v. administration of the radiolabeled conjugate 10. Metabolites of Class I are the only radiolabeled species detected. The conjugate appears to be largely intact with a small but significant amount of material converted to two species, both of which do not co-migrate with any of the controls. The relative amounts of each appear to remain constant over the course of the experiment. No Class II, II′ or III metabolites are observed in the mouse urine.

The evidence described herein demonstrates that [³²P]-labeled conjugate 10, which is chemically defined and homogeneous, is capable of crossing the cellular membrane of Hep G2 cells in a manner that is both ligand and cell-type specific. A logical extension of these investigations was to determine the tissue distribution of I in vivo and to compare the metabolic fate of 10 in vitro and in vivo and to compare the data with those obtained with conjugate 11 which lacks the three terminal GalNAc residues.

The in vivo tissue distribution data confirm the results obtained with cultured human cells. Highly selective targeting of the oligodeoxynucleoside methylphosphonate to the liver (70±10% of i.d.) was effectively achieved through covalent attachment of the oligomer and the asialoglycoprotein receptor (ASGP) ligand, YEE(ahGalNAc)₃. Indeed, the concentration of conjugate in the liver was 25-fold greater than that found in the blood and approximately 10-fold greater than in muscle based on whole tissue measurements (FIG. 11). The preference of the complex for the liver was marginal since the spleen, lungs and kidneys accumulated the radiolabeled oligo-dN as well (e.g., distribution for each tissue was about 6, 4, 2 and 2% of injected dose per gram, respectively, after 5 minutes post injection; (Lu et al., 1994, supra). It is of further interest to compare our results with those reported by Eichler et al. (1992, supra) where the biodistribution and rate of liver uptake was determined in rats for the hypolipidaemic agent ansamycin, both alone and covalently linked to another tri-anntenary ASGP ligand, N-[tris[(β-D-galactopyranosylosyl)methyl]-methyl]-N-α-(acetyl)glycinamide (tris-galacetate). The authors reported that the liver uptake of the free drug and the conjugate were roughly equivalent, leading them to conclude that the triantennary ASGP ligand did not enhance the uptake of the drug by rat liver. This result is in contrast to our finding that uptake by mouse hepatocytes is greatly facilitated by the covalent attachment of the ligand, YEE(ahGalNAc)₃.

As a control, mice were injected with conjugate 11, which lacks the three terminal GalNAc residues, and therefore should not be recognized by ASGP receptor. As anticipated, little radioactivity was detected in the liver and a far greater amount of radioactivity was associated with other tissues (FIG. 12). This result extended our previous findings that the targeting of the radiolabeled oligo-mp to hepatocytes was a consequence of its covalent attachment to the ligand.

A tritium labelled 12 mer (d-Tp*TCCTCCTGCGG) consisting of all methylphosphonate backbone except the last 5′ terminal phosphodiester linkage was injected i.v. in a single dose in mice. Organs were collected in 2, 5, 10, 30, 60 and 120 minutes after drug administration. The data shows that the radioactivity was not allocated in river, lung, muscle or spleen, and was rapidly disappearing from the plasma into the kidney and urine. The HPLC study showed that the intact 12-mer was metabolized to 11-mer via enzymatic cleavage of the terminal nucleotide and both were eliminated rapidly into the urine after i.v. injection. Thus, the results reported herein agree well with the results obtained earlier, demonstrating the importance of the GalNAc terminal in directing the uptake of oligomer conjugate into liver.

The above results were extended to consider the delivery of charged, nuclease resistant phosphorothioate oligomers to the liver of CD-1 mice. The results demonstrated that the conjugate containing the terminal sugar residues was delivered at a level more than twice that of conjugates lacking the terminal GalNAc residues. The higher concentration of the phosphorothioate containing conjugates lacking the terminal GalNAc residues in the liver as compared to ONMP's is characteristic of the phosphorothioate oligomers themselves and has been noted in the literature. However the increased delivery of these oligomers by the conjugate containing the terminal GalNAc residues to the liver illustrates the utility of this method as applied to the specific delivery of biomolecules of differing charge configurations.

In order to gain insight into the in vitro and in vivo metabolic fate of conjugate 10, we examined extracts obtained from Hep G2 cells grown in culture and from the liver and urine of mice by PAGE analysis. We noted that three classes of metabolites (Class I-III) were produced in Hep G2 cells and in mouse liver whereas only Class I metabolites were isolated from mouse urine. Class I metabolites appeared to arise owing to degradation of the ligand. Two enzymatic reactions were employed in an attempt to model the production of these species: N-acetylglucosamidase and chymotrypsin. The former treatment yielded 2, which migrated slightly faster than 1 due to the slight reduction in mass resulting from the loss of the three terminal GalNAc residues. The latter treatment resulted in a substantially enhanced mobility resulting from both the loss of a majority of the ligand and an increase in the overall charge from −1 to −2 (FIG. 16). These two model reactions produced compounds with modified ligands remaining covalently linked to intact radiolabeled oligo-mp. It is reasonable to conclude, therefore, that other species migrating to the same region of the gel resulted from degradation of the ligand and not from bond cleavage at other labile sites of I. For example, hydrolysis of a single aminohexyl side chain amide bond would yield 5 (FIG. 16) with mass between 2 and 3 and result in an increase in the negative charge from −1 to −2. In this example, a species with mobility between 2 and 3 would be expected by PAGE analysis. Class II metabolites migrate considerably faster than those identified as Class I. We propose that they arise due to unanticipated hydrolysis of the single phosphodiester linkage located at the 5′-end of the oligo-mp. It was expected that this site would be stable towards cleavage by endonuclease activity (Sproat et al., 1989, supra) based upon a model reaction conducted with snake venom phosphodiesterase in which no, cleavage was observed. Cleavage at this site would release the terminal seven nucleotides of the oligo-mp from the remainder of 10 and, most importantly, produce a relatively low molecular weight species bearing a single nucleotide containing the radiolabeled phosphate (FIG. 16; 15). Further degradation of the ligand would produce the multiple species identified as Class II metabolites. Class III metabolites, observed in Hep G2 cells only, appear to be high molecular weight species containing radioactive phosphorous that migrate a short distance into the gel. Release of radioactive phosphate from I and its subsequent incorporation into high molecular weight cellular structures (nucleic acids or proteins) would account for this band. It is well documented that the endosomal compartment acidifies as it matures, reaching pH as low as 5.5 before fusing with lysosomes (Schwartz, 1985, supra). Furthermore, the phosphoramidate linkage tying the oligo-mp to the ligand is prone to hydrolysis under acidic conditions to give 13 (FIG. 16). In order to test the possibility that acidification of the endosomal compartment resulted in the hydrolysis of the P—N bond, I was incubated at 37° C. in 50 mM sodium citrate at pH 5.5 and 6.0. We observed that I was stable at pH 6 but was substantially hydrolysed to 13 at pH 5.5 (>50%) following 24 hours and that hydrolysis occurred specifically at the phosphoramidate P—N bond as determined by PAGE analysis (data not shown). Thus, it is reasonable to conclude that incorporation of radioactive phosphate into cellular structures occurs by hydrolysis of the P—N bond due to acidification of the endosomal compartment containing I and release of the terminal phosphate into the cellular milieu by phosphatase activity.

The profile of metabolites observed in extracts from Hep G2 cells includes each class of metabolites. At early time points, the majority of the radioactivity is contained in Class I species, chiefly I and II. At later time points, the distribution of metabolites shifts from Class I to Class II and III, where at the last time point sampled, a majority of radioactive phosphorous resides with Class III metabolites, indicating substantial hydrolysis of the P—N bond had occurred over the course of the experiment. It is readily apparent that I is significantly metabolized once taken into Hep G2 cells, suggesting that intracellular delivery of an antisense oligo-mp, or other agents, would be feasible by this method.

Due to the fact that only the phosphorus at the N—P bond is labeled with ³²P, it is not possible to measure the metabolic fate of the oligonucleotide analog. Since extensive metabolism of the oligonucleotide would adversely affect the ability to specifically interact with intracellular complementary nucleic acid sequences future studies using oligonucleotide sequences labeled in other positions need to be performed.

The results of PAGE analysis of extracts obtained from mouse liver and urine demonstrate that production of metabolites in mouse liver is different from that observed with Hep G2 cells in two ways. First, digestion of 10 to produce class II′ metabolites in the liver is significantly faster, with a majority of radioactivity found in these species after only 1 hour. Second, the mobility and profile of the class II′ metabolites in the liver differs from the class II metabolites in the cultured cells, suggesting that the enzymatic activities encountered by I in mouse liver and Hep G2 cells are different. Little or no class III metabolites are produce during the 2 hour time course, a result consistent with the results from Hep G2 cells. In contrast to the extensive degradation of 1 in mouse liver, the pattern of metabolites in urine is less complex and appears to consist exclusively of Class I metabolites. The dissimilar pattern of metabolites observed for liver and urine suggest that the conjugate was delivered into liver cells and did not reside solely in the interstitial space of the organ.

The in vivo distribution and metabolism of a chemically defined, structurally homogeneous neoglycopeptide-oligodeoxynucleoside methylphosphonate conjugate 10 demonstrates that delivery of this conjugate is highly efficient, reaching levels of about 70 percent (70%) of the injected dose in the liver 15 minutes post injection. Together with the rapid and extensive degradation of the ligand, these results indicate that this method for the delivery of antisense agents, either methylphosphonates or other analogs, and other therapeutically useful agents will be very useful. Furthermore, these results demonstrate the potential for diagnostic imaging procedures that utilize the tissue specificity of the ligand coupled to the nucleic acid specificity of the antisense moiety, providing the means to measure regional abnormalities of cellular functions in vivo with heretofore unrealized specificity.

### Bioefficacy of Anti-BBV Neoglycoconjugates

### Example 13

This example illustrates the materials and methods utilized in assessing the bioefficacy of anti-HBV neoglycoconjugates.

Materials: Dulbeccos phosphate buffered saline pH 7.2, Trypsin/EDTA (0.05% Trypsin: 0.53 mm EDTA), RPMI and FCS were purchased from Mediatech, (Sterling, Va.). G418 and The Nick Translation kit were purchased from Life Technologies, (Grand Island, N.Y.). Ausyme monoclonal HBsAG detection kit was purchased from Abbott laboratories, (Napierville, Ill.). The HBsAG standard was obtained from Chemicon, (Temacia, Calif.), ³²P dCTP was obtained from Amersham, (Piscataway, N.J.) and Probequant microspin columns were purchased from Pharmacia Biotech, (Piscataway, N.J.). 48 well tissue cultured treated plates were purchased from Costar (Cambridge, Mass.), 1.5 ml microcentrifuge tubes from Sarstadt (Newton, N.C.), GeneScreen nylon membranes from NEN, (Boston, Mass.), BioTek EIA plate reader and 492 nm wavelength filter from BioTek, (Burlington, Vt.) and the Fujix Bas 1000 phosphoimager and imaging plates from Fuji Medical Systems, (Stamford, Conn.) HepG2 2.2.15 cells were a kind gift of Dr. G. Wu of and were maintained on RPMI media supplemented with 4% FCS. Cells were counted using a Coulter counter-model ZBI (Coulter Electronics, Hialeah, Fla.). The HBV specific probe (3.2 kb fragment of AM-12) was a kind gift of Dr. Brent Korba of Georgetown University.

Methods: The three therapeutic neoglycoconjugates utilized in this study were synthesized by conjugation of the following ps-oligomers, previously shown to inhibit HBV replication in vitro (Korba and Gerin, 1995, supra), to the liver specific ligand YEE(ahGalNAc)₃: (1) 5′GTTCTCCATGTTCAG3′ which targets the translation initiation site of the surface antigen gene (sa-gene), (2) 5′TTTATAAGGGTCGATGTCCAT3′ which targets the translational initiation site of the core gene (c-gene) and overlaps the HBV polyadenylation site and (3) 5′AAAGCCACCCAAGGCA3′ which targets the unpaired loop of the encapsidation site of the HBV pregenome (e-site). The base sequence used to synthesize the oligomers for this study was a HBV subtype ayw (Galibert, et al., (1979), Nature (London), 281:646-650), the same subtype expressed in vitro by HepG2 2.2.15 (Acs et al., (1987), Proc. Natl. Acad. Sci., 84:4641-4644. In addition, two additional ps-oligomers, which are non-complementary to the HBV genome, NG4: ^{5′}TGAGCTATGCACATTCAGATTT^{3′} and NG5: ⁵′TCCAATTAGATCAG³′, were prepared as controls to assay for non-specific effects of the ps-neoglycoconjugates.

Antiviral activity of the oligonucleotides was assessed using confluent cultures of Hep G2 2.2.15 cells. The HBV transfected human hepatoma cell line, Hep G2 2.2.15 was maintained on RPMI+4% fetal calf serum containing 4 mM glutamine (and incubated at 37° C., 5% CO₂ in a humidified atmosphere. Cultures were re-fed 2-3 times/week. Cells were selected with G418 and re-selected every 2-3 passages. Cells were seeded into 48-well plates at a density of 3-5×10⁴ cells/well in RPMI+2% fetal calf serum containing 4 mM glutamine and allowed to grow 34 days until confluence was achieved. At this point treatment was initiated with either neoglycoconjugates containing the above ps-oligomers or the corresponding unconjugated oligomers alone at concentrations ranging from 1.0 μM to 20 μM. Cell numbers were quantitated using a model ZB I Coulter. Confluent cultures were used due to the fact that HBV replication has been shown to reach stable, maximal levels only at this density in Hep G2 2.2.15 cells (Sells, et al., (1988), J. Virology, 62:836-844). All treatments were performed in triplicate and continued for 96 hours. Antiviral effects were assayed as detailed below and compared to untreated control cultures in order to determine the degree of inhibition. Values were reported as the average of six trials±one standard deviation.

The effect of antiviral treatment on HBV surface antigen expression (HBsAG) by Hep G2 2.2.15 cells was determined by semi-quantitative ELK analysis (Muller et. al., (1992), J. Infect. Dis., 165:929-933) using the Ausyme Monoclonal kit (Test samples were diluted so that values were in a linear dynamic range of the assay. Standard curves using HBsAG (Chemicon, Temecia, Calif.) were included in each set of analyses. Values were quantitated on a Bio-Tek EIA plate reader at a fixed wavelength of 492 nm.

Extracellular HBV DNA was analyzed by quantitative dot blot hybridization using a modification of previously described procedures (Korba and Milman, (1991), Antiviral Res., 15:217-228; Korba and Gerin, (1992), supra). Experimental and control media samples were centrifuged and treated with an equal volume of 1N NaOH-10×SSC and incubated at room temperature for 30 minutes. Samples were then applied directly to pre-soaked (0.4 Tris-HCl; pH 7.5) nylon membranes using a dot-blot apparatus. Membranes were neutralized with 0.5 M NaCl-0.5 M Tris-HCl (pH 7.5), rinsed in 2×SSC and baked at 80° C. for 2 hours.

A purified 3.2 Kb Eco R1 HBV fragment (; Korba et. al., 1989) was labeled with [³²P] dCTP using a nick translation kit and purified using ProbeQuant microspin columns. Blots were pre-hybridized for 34 hours at 42° C. in a solution containing 6×SSC, 5× Denhardts solution, 50% formamide, 0.5% SDS and 125 μg/ml denatured, sheared salmon sperm DNA. Hybridization was carried out for 18-22 h in a solution of the same composition with the addition of 10% dextran sulfate. Blots were sequentially washed at 42° C. and densitometric measurments were quantitated with a Fujix Bas 1000 phosphoimager. Virion DNA levels were determined by comparing these measurements to known amounts of HBV DNA standards applied to each blot.

### Example 14

This example illustrates the bioefficacy of liver-specific neoglyco-conjugates targeting key elements of HBV replication in Hep G2 2.2.15 cells in vitro.

In order to assess the effects of neoglycoconjugates on HBV gene expression, confluent monolayers of Hep G2 2.2.15 cells were incubated for 96 h in the presence of a single dose of either neoglycoconjugate or the corresponding oligomer alone targeting the surface antigen gene, the core gene or the encapsidation signal. The effects of these treatments on both HbsAG and HBV virion DNA accumulation in the media were assayed. Specificity of binding was confirmed by treating cells with neoglycoconjugates containing random ps-oligomers or the random ps-oligomers alone.

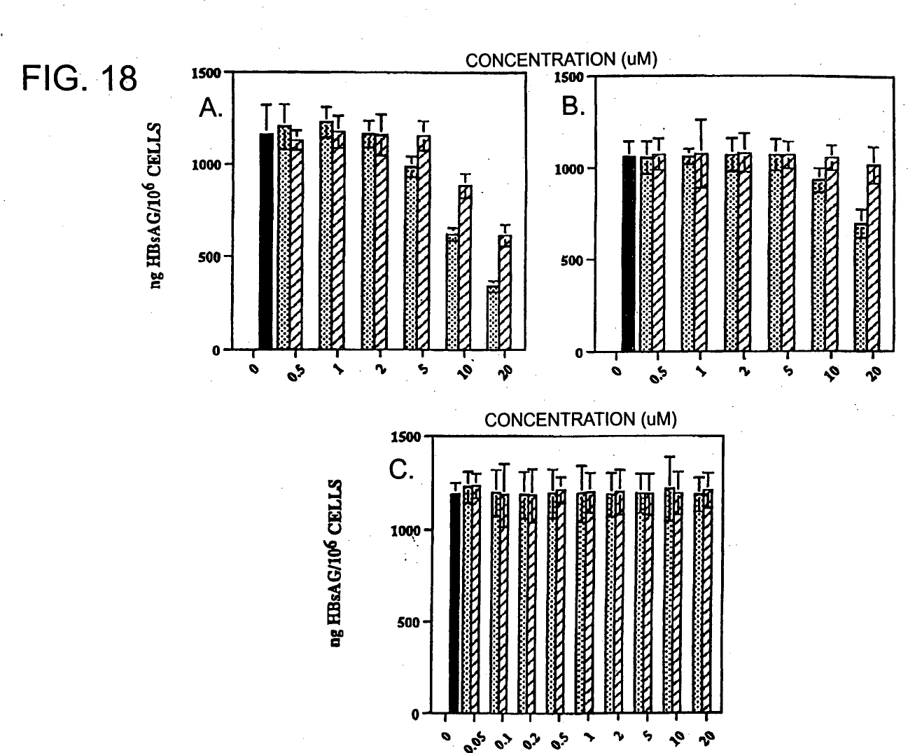

The impact of anti viral treatment upon HBsAG accumulation in the cell culture media was varible depending upon which gene sequence was targeted (FIG. 18—A. Anti-S; B. Anti-C; C. Anti-E—Solid bars=Untreated controls; Stippled bars=Neoglycoconjugates, Cross-hatched bars=unconjugated oligomer). Treatment with NG1, which targets the HBV surface antigen gene, reduced HbsAG accumulation in the media by 70% from 1163 ng/10⁶ cells to 342 ng/10⁶ at a concentration of 20 μM and by 47% to 622 ng/10⁶ cells at 10 μM. In contrast the corresponding unconjugated oligomers reduced HBsAG expression by 43% to 500 ng/10⁶ cells at 20 μM and 24% to 885 ng/10⁶ at 10 μM. Neither the neoglyconjugate or the oligomer alone had any significant effect on HBsAG expression at concentrations below 10 μM. The effect on HBV replication of neoglycoconjugates targeted against the core-gene was also examined. Treatment with NG2 resulted in a modest reduction of HBsAG from 1061 ng/10⁶ to 699.49 ng/10⁶ cells, a 35% inhibition relative to the untreated control. No significant inhibition was observed at lower concentrations. Treatment with unconjugated oligomers targeting the c-gene resulted in no significant inhibition of HbsAg. Treatment of Hep G2 2.2.15 cells with NG-3, which targets the upper stem-loop structure of the E-site, resulted in no significant reduction of HbsAG accumulation at any concentration. Similar results were observed with the corresponding unconjugated oligomer.

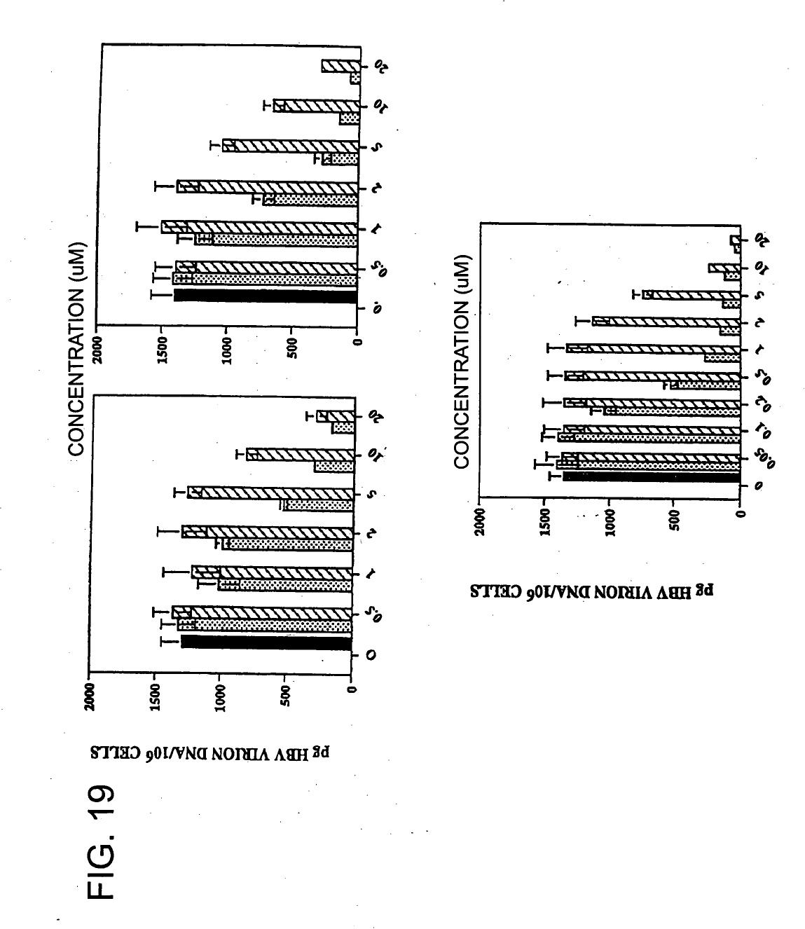

More striking was the effect of antiviral treatment on HBV virion DNA 19 accumulation in the cell culture media (FIG. 19—A. Anti-S; B. Anti-C; C. Anti-E—Solid bars=Untreated controls; Stippled bars=Neoglycoconjugates; Crosshatched Bars=Unconjugated oligomers). In this case each neoglyco-conjugate inhibited virion DNA accumulation to a significantly greater degree than the corresponding unconjugated oligomers. A dose response, was observed in all cases.

Treatment with NG3 had the greatest impact on the reduction of HBV virion DNA in the media. A greater than 80% reduction in comparison to the untreated control was observed at all concentrations down to 1. Lower concentrations of NG-3 proved to be progressively less effective until no significant inhibition was observed at 0.1 μM. Treatment with the corresponding unconjugated oligomer reduced virion DNA by <80% at concentrations of 20 and 10 μM respectively. At lower concentrations virion DNA levels progressively increased until untreated control levels were reached at concentrations between 1-2 μM.

NG-2, which targets the core gene reduced virion DNA accumulation significantly, but to a lesser degree than NG-3. In this case DNA was reduced by more than 80% down to a concentration of 5 μM. The corresponding unconjugated oligomer also reduced virion DNA production by greater than 80% at a concentration of 20 μM. However thereafter the neoglycoconjugate proved to be approximately 4 times more effective at all concentrations until untreated control levels were reached at 1 μM.

Treatment with NG-1 also resulted in a significant decrease of virion DNA in the cell culture media. Levels were decreased in comparison to the untreated control by more than 70% to a concentration of 10 μM. At lower treatment concentrations, the virion DNA levels increased until control levels were reached between 1-2 μM. Again, the unconjugated oligomer suppressed virion DNA levels to a similar degree at a concentration of 20 μM. However, the neoglycoconjugate proved to be 4-5 times more effective at all concentrations thereafter down to 2 μM.

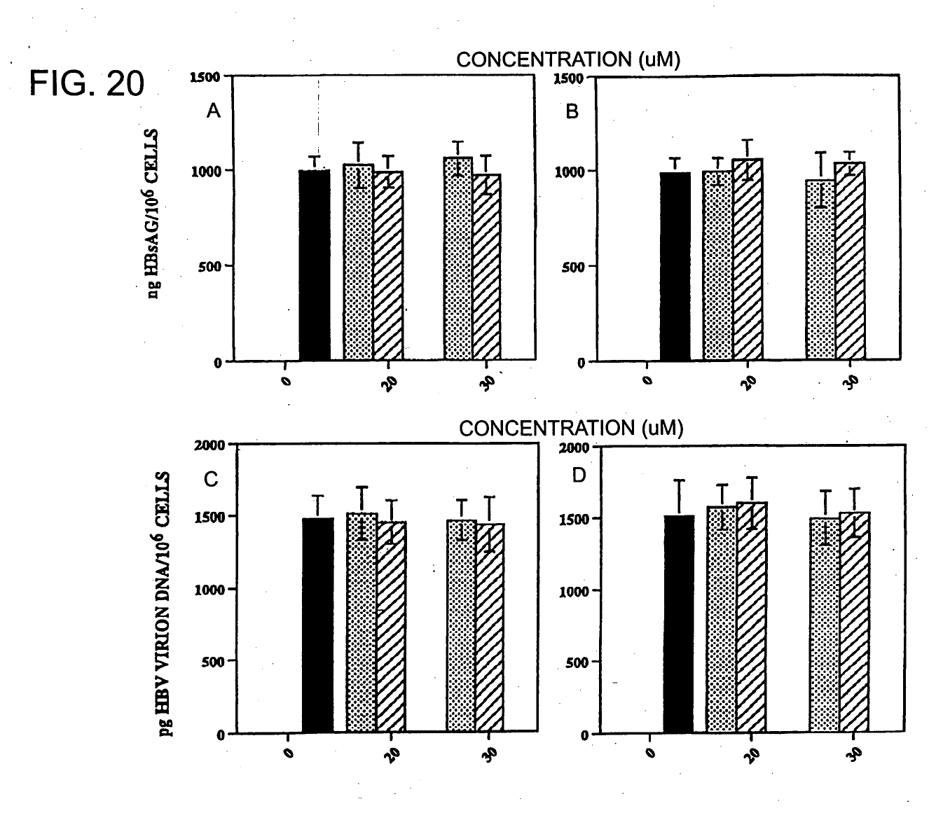

In order to confirm the specificity of the above treatments, random ps-oligomers non-complementary to any portion of the HBV genome were synthesized (NG4 and NG5). Treatment with NG4 and NG5 in either oligomer or neoglyco-conjugate form had no significant effect on HBsAG or virion DNA accumulation in the cell culture media at concentrations up to 30 μM (FIG. 20—A=Effect of NG-4 on HBs AG accumulation; B=Effect of NG-5 and corresponding oligomer on HBsAG accumulation; C. Effect of NG4 and corresponding oligomer on HBV virion DNA accumulation; D. Effect of NG-5 corresponding oligomer on HBV virion DNA accumulation. Solid bars=untreated controls, Stippled bars=neoglycoconjugate; Cross-hatched bars=unconjugated oligomers).

### Example 15

This example illustrates stability studies of phosphorothioate neoglycoconjugates in cell culture media

The in vitro stability of the neoglycoconjugates and unconjugated phosphorothioate oligomers used in this study was determined by PAGE analysis. Neoglycoconjugates NGI-5 and their unconjugated forms were incubated in RPMI+2% FCS at 37° C. for 24, 48, and 96 hours. Aliquots containing 2 μl of either neoglycoconjugates or unconjugated oligomers were added to 10 μl of gel loading buffer and electrophoresed for 20 minutes at 800 V on a 20% polyacrylamide gel containing 7 M urea. The resulting gels were analyzed with a Fujix Bas 1000 phosphoimager (Fuji Medical Systems, Stamford, Conn.). All neoglycoconjugates and oligomers remained intact in cell culture media for up to 96 hours.

### Example 16

This example illustrates the toxicity analysis of the phosphorothioate neoglycoconjugates.

The toxicity of each treatment used in this study was determined by Trypan Blue exclusion. Measurements were made under culture conditions used for the antiviral experiments. No significant toxicity at any concentration for any treatment was noted. After the treatment period, the number of viable cells was determined by microscopically by Trypan Blue exclusion. A minimum of at least 200 cells from each well were counted. All determinations were performed on triplicate wells.

The invention may be embodied in other specific forms without departing from the spirit or essential characteristics thereof. The present embodiments are therefore to be considered in all respects as illustrative and not restrictive. Thus, it is understood that a large variety of compounds can be synthesized using the methods described herein. All methods described herein can be performed in any suitable order unless otherwise indicated herein or otherwise clearly contradicted by context. Patents, patent applications, and other literature cited herein are hereby fully incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention. This invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A construct comprising a homogeneous conjugate of formula A-L-P, wherein
A represents a hepatic ligand that specifically binds to a hepatic receptor, thereby facilitating the entrance of said conjugate into cells having said receptor;
L represents a bifunctional linker that is covalently linked to A in a regiospecific manner to form A-L; A-L is covalently linked to P in a regiospecific manner to form A-L-P;
P represents a biologically stable oligomer comprising the nucleotide sequence of SEQ ID NO:27 or SEQ ID NO:28, wherein P is released from the conjugate following hydrolysis or reduction of at least one specific biochemical linkage, and contains internucleotide linkages resistant to enzymatic hydrolysis of biodegradation upon release from the conjugate.

2. The construct of claim 1, wherein said oligomer is an oligonucleotide, an oligonucleotide analog or an oligonucleoside.

4.-17. (canceled)

18. The construct of claim 2, wherein said oligomer comprises deoxyribose methylphosphonate internucleotide linkages.

19. The construct of claim 2, wherein said oligomer comprises deoxyribose phosphorothioate internucleotide linkages.

20. The construct of claim 2, wherein said oligomer comprises phosphodiester linkages.

21. The construct of claim 2, wherein said oligomer comprises a combination of deoxyribose methylphosphonate/phosphorothioate internucleotide linkages.

22. The construct of claim 2, wherein said oligomer comprises a combination of deoxyribose methylphosphonate/phosphodiester internucleotide linkages.

23. The construct of claim 2, wherein said oligomer comprises deoxyribose phosphorothioate/phosphodiester internucleotide linkages.

24. The construct of claim 2, wherein said oligomer comprises 2′-O-methylribose methylphosphonate internucleotide linkages.

25. The construct of claim 2, wherein said oligomer comprises 2′-O-methylribose phosphorothioate internucleotide linkages.

26. The construct of claim 2, wherein said oligomer comprises 2′-O-methylribose phosphodiester internucleotide linkages.

27. The construct of claim 2, wherein said oligomer comprises a combination of 2′-O-methylribose methylphosphonate/2′-O-methylribose phosphodiester internucleotide linkages.

28. The construct of claim 2, wherein said oligomer comprises a combination of 2′-O-methylribose methylphosphonate/2′-O-methylribose phosphorothionate internucleotide linkages.

29. The construct of claim 2, wherein said oligomer comprises a combination of 2′-O-methylribose phosphorothioate/2′-O-methylribose phosphodiester internucleotide linkages.

30.-63. (canceled)

64. A pharmaceutical composition comprising a construct according to claim 1 and at least one pharmaceutically acceptable excipient or carrier.

65.-69. (canceled)

70. The pharmaceutical composition of claim e 64 wherein the A-L moiety of said construct is YEE(ahGalNAc)₃-SMCC.

71. (canceled)

72. The construct of claim 1, wherein the A-L moiety of said construct is YEE(ahGalNAc)₃-SMCC.

73. (canceled)
